(19) United States
(12) Patent Application Publication
(10) Pub. No.: US 2003/0166633 A1
(43) Pub. Date: Sep. 4, 2003

(54) Pyridinylimidazoles

(76) Inventors: Laramie Mary Gaster, Hertfordshire (GB); Michael Stewart Hadley, Hertfordshire (GB); John David Harling, Hertfordshire (GB); Frank Peter Harrington, Hertfordshire (GB); Jag Paul Heer, Essex (GB); Thomas Daniel Heightman, Hertfordshire (GB); Andrew Hele Payne, Hertfordshire (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/204,370

(22) PCT Filed: Feb. 21, 2001
(86) PCT No.: PCT/GB01/00736

(30) Foreign Application Priority Data
Feb. 21, 2000 (GB) 0004053.5
Jun. 28, 2000 (GB) 0015902.0

(51) Int. Cl. C07D 417/14; C07D 413/14; C 07D 4 3/14; A61K 31/551; A61K 31/541; A61K 31/553; A61K 31/554; A61K 31/5377; A61K 31/496; A61K 31/4545; A61K 31/4439

(52) U.S. Cl. 514/211.15; 514/217.04; 514/218; 514/341; 514/227.8; 514/235.8; 514/253.09; 514/318; 540/544; 540/575; 540/597; 544/60; 544/360; 544/124; 546/193; 546/272.7

(57) ABSTRACT

Compounds of formula (I) and pharmaceutically acceptable salts thereof wherein R₁, R₂ and R₃ represent various functional groups, and one of X₁ and X₂ is N and the other is NR₁₀; and their use as pharmaceuticals.

This invention relates to pyridyl substituted imidazoles which are inhibitors of the transforming growth factor, (“TGF”)-β signaling pathway, in particular, the phosphorylation of smad2 or smad3 by the type I or activin-like kinase (“ALK”)-5 receptor, methods for their preparation and their use in medicine, specifically in the treatment and prevention of a disease state mediated by this pathway.

TGF-β1 is the prototypic member of a family of cytokines including the TGF-βs, activins, inhibins, bone morphogenetic proteins and Mullerian-inhibiting substance, that signal through a family of single transmembrane serine/threonine kinase receptors. These receptors can be divided in two classes, the type I or activin like kinase (ALK) receptors and type II receptors. The ALK receptors are distinguished from the type II receptors in that the ALK receptors (a) lack the serine/threonine rich intracellular tail, (b) possess serine/threonine kinase domains that are very homologous between type I receptors, and (c) share a common sequence motif called the GS domain, consisting of a region rich in glycine and serine residues. The GS domain is at the amino terminal end of the intracellular kinase domain and is critical for activation by the type II receptor. Several studies have shown that TGF-β, signaling requires both the ALK and type II receptors. Specifically, the type II receptor phosphorylates the GS domain of the type I receptor for TGF-β, ALK5, in the presence of TGF-β. The ALK5, in turn, phosphorylates the cytoplasmic proteins smad2 and smad3 at two carboxy terminal serines. Generally it is believed that in many species, the type II receptors regulate cell proliferation and the type I receptors regulate matrix production. Therefore, preferred compounds of this invention are selective in that they inhibit the type I receptor and thus matrix production, and not the type II receptor mediated proliferation.

Activation of the TGF-β1 axis and expansion of extracellular matrix are early and persistent contributors to the development and progression of chronic renal disease and vascular disease. Border W. A., Noble N. A., N. Engl. J. Med., Nov. 10, 1994; 331(19):1286-92. Further, TGF-β1 plays a role in the formation of fibronectin and plasminogen activator inhibitor-1, components of sclerotic deposits, through the action of smad3 phosphorylation by the TGF-β1 receptor ALK5. Zhang Y., Feng X. H., Derynck R., Nature, Aug. 27, 1998; 394(6696):909-13; Usui T., Takase M., Kaji Y., Suzuki K., Ishida K., Tsuru T., Miyata K., Kawabata M., Yamashita H., Invest. Ophthalmol. Vis. Sci., October 1998; 39(11):1981-9.

Progressive fibrosis in the kidney and cardiovascular system is a major cause of suffering and death and an important contributor to the cost of health care. TGF-β1 has been implicated in many renal fibrotic disorders. Border W. A., Noble N. A., N. Engl. J. Med., Nov. 10, 1994; 331(19): 1286-92. TGF-β1 is elevated in acute and chronic glomerulonephritis, Yoshioka K., Takemura T., Murakami K., Okada M., Hino S., Miyamoto H., Maki S., Lab. Invest., February 1993; 68(2):154-63, diabetic nephropathy, Yamamoto, T., Nakamura, T., Noble, N. A., Ruoslahti, E., Border, W. A., (1993) PNAS 90:1814-1818, allograft rejection, HIV nephropathy and angiotensin-induced nephropathy, Border W. A., Noble N. A., N. Engl. J. Med., Nov. 10, 1994; 331(19): 1286-92. In these diseases the levels of TGF-β1 expression coincide with the production of extracellular matrix. Three lines of evidence suggest a causal relationship between TGF-β1 and the production of matrix. First, normal glomeruli, mesangial cells and non-renal cells can be induced to produce extracellular-matrix protein and inhibit protease activity by exogenous TGF-β1 in vitro. Second, neutralizing anti-bodies against TGF-β1 can prevent the accumulation of extracellular matrix in nephritic rats. Third, TGF-β1 transgenic mice or in vivo transfection of the TGF-β1 gene into normal rat kidneys resulted in the rapid development of glomerulosclerosis. Kopp J. B., Factor V. M., Mozes M., Nagy P., Sanderson N., Bottinger E. P., Klotman P. E., Thorgeirsson S. S., Lab Invest, June 1996; 74(6):991-1003. Thus, inhibition of TGF-β1 activity is indicated as a therapeutic intervention in chronic renal disease.

TGF-β1 and its receptors are increased in injured blood vessels and are indicated in neointima formation following balloon angioplasty, Saltis J., Agrotis A., Bobik A., Clin Exp Pharmacol Physiol, March 1996; 23(3):193-200. In addition TGF-β1 is a potent stimulator of smooth muscle cell (“SMC”) migration in vitro and migration of SMC in the arterial wall is a contributing factor in the pathogenesis of atherosclerosis and restenosis. Moreover, in multivariate analysis of the endothelial cell products against total cholesterol, TGF-β receptor ALK5 correlated with total cholesterol (P<0.001) Blann A. D., Wang J. M., Wilson P. B., Kumar S., Atherosclerosis, February 1996; 120(1-2):221-6. Furthermore, SMC derived from human atherosclerotic lesions have an increased ALK5/TGF-β type II receptor ratio. Because TGF-β1 is over-expressed in fibroproliferative vascular lesions, receptor-variant cells would be allowed to grow in a slow, but uncontrolled fashion, while overproducing extracellular matrix components McCaffrey T. A., Consigli S., Du B., Falcone D. J., Sanborn T. A., Spokojny A. M., Bush H. L., Jr., J Clin Invest, December 1995; 96(6):2667-75. TGF-β1 was immunolocalized to non-foamy macrophages in atherosclerotic lesions where active matrix synthesis occurs, suggesting that non-foamy macrophages may participate in modulating matrix gene expression in atherosclerotic remodeling via a TGF-β1-dependent mechanism. Therefore, inhibiting the action of TGF-β1 on ALK5 is also indicated in atherosclerosis and restenosis.

TGF-β is also indicated in wound repair. Neutralizing antibodies to TGF-β1 have been used in a number of models to illustrate that inhibition of TGF-β1signaling is beneficial in restoring function after injury by limiting excessive scar formation during the healing process. For example, neutralizing antibodies to TGF-β1 and TGF-β2 reduced scar formation and improved the cytoarchitecture of the neodermis by reducing the number of monocytes and macrophages as well as decreasing dermal fibronectin and collagen deposition in rats Shah M., J. Cell. Sci., 1995, 108, 985-1002. Moreover, TGF-β antibodies also improve healing of corneal wounds in rabbits Moller-Pedersen T., Curr. Eye Res., 1998, 17, 736-747, and accelerate wound healing of gastric ulcers in the rat, Ernst H., Gut, 1996, 39, 172-175. These data strongly suggest that limiting the activity of TGF-β would be beneficial in many tissues and suggest that any disease with chronic elevation of TGF-β would benefit by inhibiting smad2 and smad3 signaling pathways.

TGF-β is also implicated in peritoneal adhesions Saed G. M., et al, Wound Repair Regeneration, 1999 November-December, 7(6), 504-510. Therefore, inhibitors of ALK5 would be beneficial in preventing peritoneal and sub-dermal fibrotic adhesions following surgical procedures.

TGFβ1-antibodies prevent transplanted renal tumor growth in nude mice through what is thought to be an anti-angiogenic mechanism Ananth S, et al, Journal Of The American Society Of Nephrology Abstracts, 9: 433A(Abstract). While the tumor itself is not responsive to TGF-β the surrounding tissue is responsive and supports tumor growth by neovascularization of the TGF-β secreting tumor. Thus, antagonism of the TGF-β pathway should prevent metastasis growth and reduce cancer burden.

Bioorg. Med. Chem. Lett., 1995, 5(6), 543 discloses 2-[5-(2-methylphenyl)-2-propyl-1H-imidazol-4-yl]pyridine as an inhibitor of gastric H⁺/K⁺ ATPase.

DE 2221546 discloses the following compounds as antiinflammatory, analgesic or antipyretic agents:

2-[2-(1,1-dimethylethyl)-5-(4-methoxyphenyl)-1H-imidazol-4-yl]pyridine,

2-[2-(1,1-dimethylethyl)-5-phenyl-1H-imidazol-4-yl]pyridine.

Japanese Patent No. 09124640 discloses the following compounds as agrochemical fungicides:

2-[5-(3,5-dichlorophenyl)-2-methyl-1H-imidazol-4-yl]pyridine,

2-[5-(3,5-dimethylphenyl)-2-methyl-1H-imidazol-4-yl]pyridine,

2-[5-(3,5-dimethylphenyl)-2-ethyl-1H-imidazol-4-yl]pyridine,

2-[5-(3,5-dimethylphenyl)-2-amino-1H-imidazol-4-yl]pyridine,

2-[5-(3,5-dimethylphenyl)-2-isopropyl-1H-imidazol-4-yl]pyridine,

2-[5-(3,5-dimethylphenyl)-2-propyl-1H-imidazol-4-yl]pyridine,

2-[5-(3,5-dimethylphenyl)-2-carboxamide-1H-imidazol-4-yl]pyridine.

Surprisingly, it has now been discovered that a class of 2-pyridyl substituted imidazoles of formula (I), function as potent and selective non-peptide inhibitors of ALK5 kinase and therefore, have utility in the treatment and prevention of various disease states mediated by ALK5 kinase mechanisms, such as chronic renal disease, acute renal disease, wound healing, arthritis, osteoporosis, kidney disease, congestive heart failure, ulcers, ocular disorders, corneal wounds, diabetic nephropathy, impaired neurological function, Alzheimer's disease, trophic conditions, atherosclerosis, peritoneal and sub-dermal adhesion, any disease wherein fibrosis is a major component, including, but not limited to lung fibrosis and liver fibrosis, and restenosis.

According to the invention there is provided a compound of formula (I) or a pharmaceutically acceptable salt thereof:

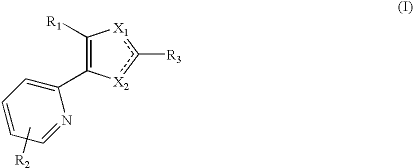

wherein R₁ is naphthyl, anthracenyl, or phenyl optionally substituted with one or more substituents selected from the group consisting of halo, C₁₋₆alkoxy, C₁₋₆alkylthio, C₁₋₆alkyl, C₁₋₆haloalkyl, O—(CH₂)ₘ—Ph, S—(CH₂)ₘ—Ph, cyano, phenyl, and CO₂R, wherein R is hydrogen or C₁₋₆alkyl and m is 0-3; or R₁ is phenyl or pyridyl fused with an aromatic or non-aromatic cyclic ring of 5-7 members wherein said cyclic ring optionally contains up to three heteroatoms, independently selected from N, O and S, and is optionally substituted by ═O;

R₂ represents hydrogen, C₁₋₆alkyl, C₁₋₆ alkoxy, phenyl, C₁₋₆haloalkyl, halo, NH₂, NH—C₁₋₆alkyl or NH(CH₂)ₙ—Ph wherein n is 0-3;

R₃ represents C₁₋₆alkyl, —(CH₂)ₚ—CN, —(CH₂)ₚ—COOH, —(CH₂)ₚ—CONHR₄R₅, —(CH₂)ₚCOR₄, —(CH₂)_{q}(OR₆)₂, —(CH₂)ₚOR₄, —(CH₂)_{q}CH═CH—CN, —(CH₂)_{q}—CH═CH—CO₂H, —(CH₂)ₚ—CH═CH—CONHR₄R₅, —(CH₂)ₚNHCOR₇ or —(CH₂)ₚNR₈R₉,

R₄ and R₅ are independently hydrogen or C₁₋₆alkyl;

R₆ is C₁₋₆alkyl;

R₇ is C₁₋₇alkyl, or optionally substituted aryl, heteroaryl, arylC₁₋₆alkyl or heteroarylC₁₋₆alkyl;

R₈ and R₉ are independently selected from hydrogen, C₁₋₆alkyl, aryl and arylC₁₋₆alkyl;

p is 0-4;

q is 1-4;

one of X₁ and X₂ is N and the other is NR₁₀; and

R₁₀is hydrogen, C₁₋₆alkyl, or C₃₋₇cycloalkyl;

provided that the compound is not:

i) 2-[5-(2-methylphenyl)-2-propyl-1H-imidazol-4-yl]pyridine,

ii) 2-[2-(1,1-dimethylethyl)-5-(4-methoxyphenyl)-1H-imidazol-4-yl]pyridine,

iii) 2-[2-(1,1-dimethylethyl)-5-phenyl-1H-imidazol-4-yl]pyridine,

iv) 2-[5-(3,5-dichlorophenyl)-2-methyl-1H-imidazol-4-yl]pyridine,

v) 2-[5-(3,5-dimethylphenyl)-2-methyl-1H-imidazol-4-yl]pyridine,

vi) 2-[5-(3,5-dimethylphenyl)-2-ethyl-1H-imidazol-4yl]pyridine,

vii) 2-[5-(3,5-dimethylphenyl)-2-amino-1H-imidazol-4-yl]pyridine,

viii) 2-[5-(3,5-dimethylphenyl)-2-isopropyl-1H-imidazol-4-yl]pyridine,

ix) 2-[5-(3,5-dimethylphenyl)-2-propyl-1H-imidazol-4-yl]pyridine, or

x) 2-[5-(3,5-dimethylphenyl)-2-carboxamide-1H-imidazol-4-yl]pyridine.

As used herein, the double bond indicated by the dotted lines of formula (I), represent the possible tautomeric ring forms of the compounds falling within the scope of this invention, the double bond being to the unsubstituted nitrogen.

In a preferred group of compounds R₁ is optionally substituted naphthyl or phenyl. Preferably R₁ is phenyl optionally substituted with one or more substituents selected from the group consisting of halo, C₁₋₆alkoxy, C₁₋₆alkylthio, and phenyl; more preferably R₁ is phenyl optionally substituted with one or more substituents selected from the group consisting of halo, C₁₋₆alkoxy, C₁₋₆alkylthio, and cyano; or R₁ is phenyl or pyridyl (notably phenyl) fused with an aromatic or non-aromatic cyclic ring of 5-7 members wherein said cyclic ring optionally contains up to three heteroatoms, independently selected from N, O and S, and is optionally substituted by ═O; for example R₁ represents benzo[1,3]dioxolyl, 2,3-dihydrobenzo[1,4]dioxinyl, benzoxazolyl, benzothiazolyl, quinoxalinyl, benzo[1,2,5]oxadiazolyl, benzo[1,2,5]thiadiazolyl, [1,2,4]triazolo[1,5-a]pyridyl, dihydrobenzofuranyl, benzo[1,4]oxazinyl-3-one or benzoxazolyl-2-one.

Preferably R₂ is other than hydrogen. When R₂ is other than hydrogen it is preferably positioned ortho to the nitrogen of the pyridyl ring.

Preferably R₃ is C₁₋₆alkyl or (CH₂)ₚNHCOR₇ wherein R₇ is C₁₋₇alkyl, or optionally substituted aryl, heteroaryl, aryl C₁₋₆alkyl or heteroaryl C₁₋₆alkyl.

Preferably one of X₁ and X₂ is N and the other is NR₁₀, wherein R₁₀ is hydrogen or C₁₋₆alkyl.

R₁₀ is preferably hydrogen.

The compounds for use in the methods of the invention preferably have a molecular weight of less than 800, more preferably less than 600.

Specific compounds of the invention which may be mentioned include those described in the examples.

Suitable, pharmaceutically acceptable salts of the compounds of formula (I) include, but are not limited to, salts with inorganic acids such as hydrochloride, sulfate, phosphate, diphosphate, hydrobromide, and nitrate, or salts with an organic acid such as malate, maleate, fumarate, tartrate, succinate, citrate, acetate, lactate, methanesulfonate, p-toluenesulfonate, palmitate, salicylate, and stearate.

Some of the compounds of this invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

Certain of the compounds of formula (I) may exist in the form of optical isomers, e.g. diastereoisomers and mixtures of isomers in all ratios, e.g. racemic mixtures. The invention includes all such forms, in particular the pure isomeric forms. The different isomeric forms may be separated or resolved one from the other by conventional methods, or any given isomer may be obtained by conventional synthetic methods or by stereospecific or asymmetric syntheses.

Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably at least 10% of a compound of formula (I) or pharmaceutically acceptable derivative thereof.

The terms “C₁₋₆alkyl” and “C₁₋₇alkyl” as used herein whether on its own or as part of a larger group e.g. C₁₋₆alkoxy, means a straight or branched chain radical of 1 to 6 and 1 to 7 carbon atoms respectively, including, but not limited to methyl, ethyl, n-propyl, isopropyl, n-butyl, sec-butyl, isobutyl and tert-butyl.

C₁₋₆haloalkyl groups may contain one or more halo atoms, a particular C¹⁻⁶ haloalkyl group that may be mentioned in CF₃.

The terms “halo” or “halogen” are used interchangeably herein to mean radicals derived from the elements chlorine, fluorine, iodine and bromine.

The term “C₃₋₇cycloalkyl” as used herein means cyclic radicals of 3 to 7 carbons, including but not limited to cyclopropyl, cyclopentyl and cyclohexyl.

The term “aryl” as used herein means 5- to 14-membered substituted or unsubstituted aromatic ring(s) or ring systems which may include bi- or tri-cyclic systems, including, but not limited to phenyl and naphthyl.

The term “ALK5 inhibitor” as used herein means a compound, other than inhibitory smads, e.g. smad6 and smad7, which selectively inhibits the ALK5 receptor preferentially over p38 or type II receptors.

The term “ALK5 mediated disease state” as used herein means any disease state which is mediated (or modulated) by ALK5, for example a disease which is modulated by the inhibition of the phosphorylation of smad 2/3 in the TGF-1β signaling pathway.

The term “ulcers” as used herein includes, but is not limited to, diabetic ulcers, chronic ulcers, gastric ulcers, and duodenal ulcers.

The compounds of formula (I) can be prepared by art-recognized procedures from known or commercially available starting materials. If the starting materials are unavailable from a commercial source, their synthesis is described herein, or they can be prepared by procedures known in the art.

Specifically, compounds of formula (I) where one of X₁ and X₂ is NH may be prepared according to Scheme 1. The ketone may be oxidised to a diketone with HBr in DMSO. This diketone can then be condensed with a suitably substituted aldehyde or protected aldehyde derivative and ammonium acetate to give the imidazole according to the method outlined in WO 98/56788. Alternatively the ketone may be treated with sodium nitrite in HCI to afford an α-oximinoketone which can then be condensed with a suitably substituted aldehyde or protected aldehyde derivative and ammonium acetate to give the N-hydroxyimidazole. Treatment of this with triethylphosphite affords the imidazole according to the method outlined in U.S. Pat. No. 5,656,644.

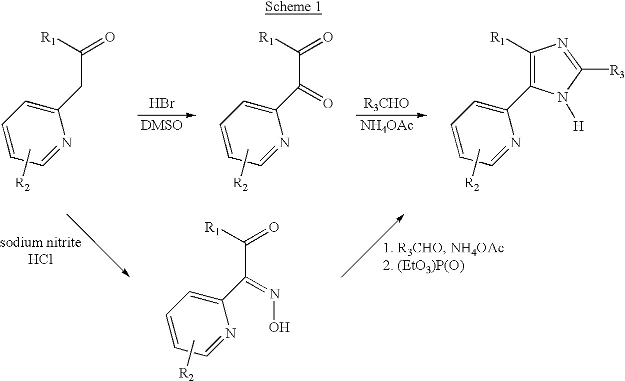

Compounds of formula (I) where one of X₁ and X₂ is NH may also be prepared according to Scheme 2. A suitable bromide is coupled with trimethylsilylacetylene using palladium catalysis. The trimethylsilyl group can be removed by treatment with potassium carbonate and the terminal acetylene coupled with 6-bromo-2-methylpyridine again using palladium catalysis. The acetylene may then be oxidised to the diketone using palladium chloride in DMSO. Formation of the imidazole is then carried out with a suitable aldehyde as described in Scheme 1.

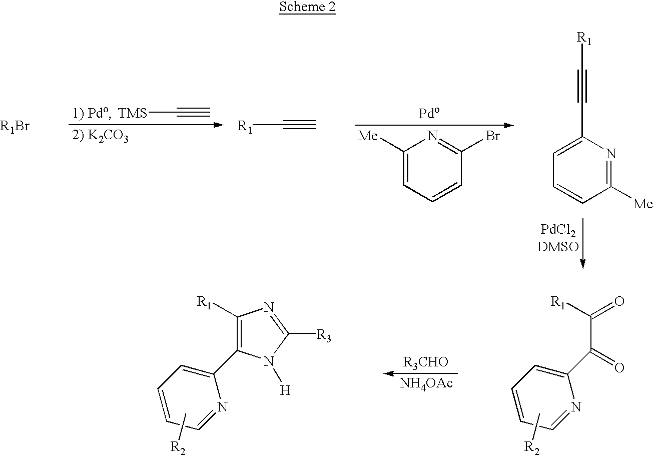

Non-selective alkylation of the imidazole nitrogen (using one of the procedures outlined in N. J. Liverton et al; J. Med. Chem., 1999, 42, 2180-2190) with a compound of formula L—R₁₀ wherein L is a leaving group, e.g. halo, sulfonate or triflate, will yield both isomers of the compounds where X₁ or X₂ is NR₁₀ in which R₁₀ is other than hydrogen, the isomers can be separated by chromatographic methods (Scheme 3).

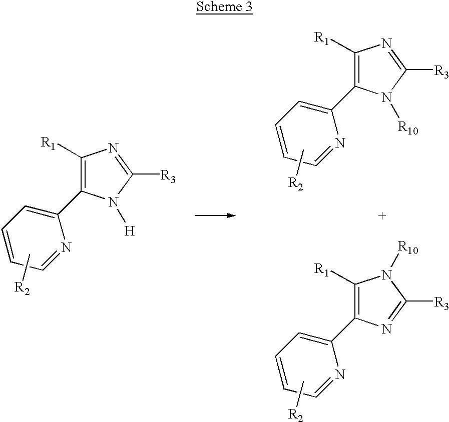

Compounds of formula (I) where R₃ is —CH₂NHCOR₇ may be prepared according to Scheme 4. The appropriate dione is condensed with (1,3-dioxo-1,3-dihydro-isoindol-2-yl)-acetaldehyde and ammonium acetate to form the imidazole. This product is treated with hydrazine to unmask the free amine which can then be coupled to an appropriate carboxylic acid using standard amide bond formation conditions.

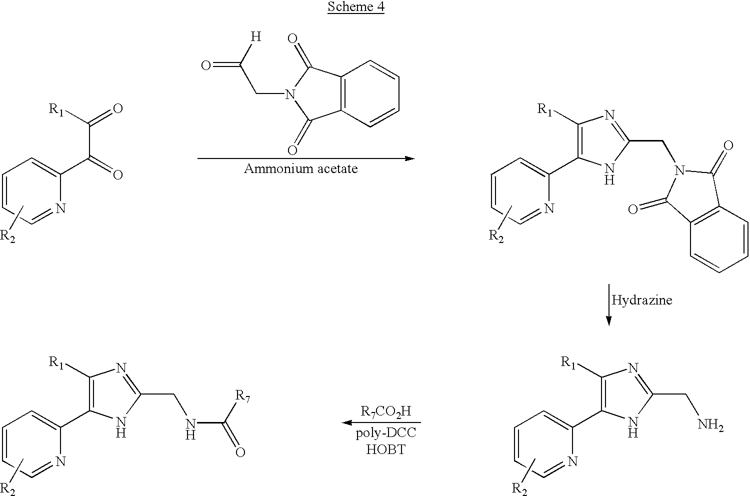

During the synthesis of the compounds of formula (I) labile functional groups in the intermediate compounds, e.g. hydroxy, carboxy and amino groups, may be protected. A comprehensive discussion of the ways in which various labile functional groups may be protected and methods for cleaving the resulting protected derivatives is given in for example Protective Groups in Organic Chemistry, T. W. Greene and P. G. M. Wuts, (Wiley-Interscience, New York, 2nd edition, 1991).

Further details for the preparation of compounds of formula (I) are found in the examples.

The compounds of formula (I) may be prepared singly or as compound libraries comprising at least 2, for example 5 to 1,000 compounds, and more preferably 10 to 100 compounds of formula (I). Libraries of compounds of formula (I) may be prepared by a combinatorial ‘split and mix’ approach or by multiple parallel synthesis using either solution phase or solid phase chemistry, by procedures known to those skilled in the art.

Thus according to a further aspect of the invention there is provided a compound library comprising at least 2 compounds of formula (I) or pharmaceutically acceptable salts thereof.

The invention further provides the use of a compound of formula (I), but without provisos i) to x), or a pharmaceutically acceptable salt thereof, in the manufacture of a medicament for the treatment of a disease mediated by the ALK5 receptor in mammals.

The invention further provides a method of treatment of a disease mediated by the ALK5 receptor in mammals, comprising administering to a mammal in need of such treatment, a therapeutically effective amount of a compound of formula (I), but without provisos i) to x), or a pharmaceutically acceptable salt thereof.

ALK5-mediated disease states, include, but are not limited to, chronic renal disease, acute renal disease, wound healing, arthritis, osteoporosis, kidney disease, congestive heart failure, ulcers, ocular disorders, corneal wounds, diabetic nephropathy, impaired neurological function, Alzheimer's disease, trophic conditions, atherosclerosis, any disease wherein fibrosis is a major component, including, but not limited to peritoneal and sub-dermal adhesion, lung fibrosis and liver fibrosis, and restenosis.

By the term “treating” is meant either prophylactic or therapeutic therapy.

The invention further provides a method of inhibiting the TGF-β signaling pathway in mammals, for example, inhibiting the phosphorylation of smad2 or smad3 by the type I or activin-like kinase ALK5 receptor, which method comprises administering to a mammal in need of such treatment, a therapeutically effective amount of a compound of formula (I), but without provisos i) to x), or a pharmaceutically acceptable salt thereof.

The invention further provides the use of a compound of formula (I,) but without provisos i) to x), or a pharmaceutically acceptable salt thereof, in the manufacture of a medicament for inhibiting the TGF-β signaling pathway in mammals.

The invention further provides a method of inhibiting matrix formation in mammals, for example, by inhibiting the phosphorylation of smad2 or smad3 by the type I or activin-like kinase ALK5 receptor, which method comprises administering to a mammal in need of such treatment, a therapeutically effective amount of a compound of formula (I), but without provisos i) to x), or a pharmaceutically acceptable salt thereof.

The invention further provides the use of a compound of formula (I), but without provisos i) to x), or a pharmaceutically acceptable salt thereof, in the manufacture of a medicament for inhibiting matrix formation in mammals.

The compounds of formula (I) and pharmaceutically acceptable salts thereof, may be administered in conventional dosage forms prepared by combining a compound of formula (I), but without provisos i) to x), with standard pharmaceutical carriers or diluents according to conventional procedures well known in the art. These procedures may involve mixing, granulating and compressing or dissolving the ingredients as appropriate to the desired preparation.

According to a further aspect of the present invention there is provided a pharmaceutical composition comprising a compound of formula (I), but without provisos iv) to x), or a pharmaceutically acceptable salt thereof, and a pharmaceutically acceptable carrier or diluent.

The pharmaceutical compositions of the invention may be formulated for administration by any route, and include those in a form adapted for oral, topical or parenteral administration to mammals including humans.

The compositions may be formulated for administration by any route. The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material, depending on the method of administration. Where the compositions comprise dosage units, each unit will preferably contain from 50-500 mg of the active ingredient. The dosage as employed for adult human treatment will preferably range from 100 to 3000 mg per day, for instance 1500 mg per day depending on the route and frequency of administration. Such a dosage corresponds to 1.5 to 50 mg/kg per day. Suitably the dosage is from 5 to 20 mg/kg per day.

It will be recognized by one of skill in the art that the optimal quantity and spacing of individual dosages of a compound of formula (I), but without provisos i) to x), will be determined by the nature and extent of the condition being treated, the form, route and site of administration, and the particular mammal being treated, and that such optimums can be determined by conventional techniques. It will also be appreciated by one of skill in the art that the optimal course of treatment, i.e. the number of doses of the compound of formula (I), but without provisos i) to x), given per day for a defined number of days, can be ascertained by those skilled in the art using conventional course of treatment determination tests.

No toxicological effects are indicated when a compound of formula (I), but without provisos i) to x), or a pharmaceutically acceptable salt thereof is administered in the above-mentioned dosage range.

All publications, including, but not limited to, patents and patent applications cited in this specification, are herein incorporated by reference as if each individual publication were specifically and individually indicated to be incorporated by reference herein as though fully set forth.

The following examples are to be construed as merely illustrative and not a limitation on the scope of the invention in any way. In the Examples, mass spectra were performed using an Hitachi Perkin-Elmer RMU-6E with chemical ionization technique (CI) or a Micromass Platform II instrument with electrospray (ES) ionization technique.

### EXAMPLE

### Description 1

### 1-Benzo[1,3]dioxol-5-yl-2-(6-methyl-pyridin-2-yl)-ethane-1,2-dione (D1)

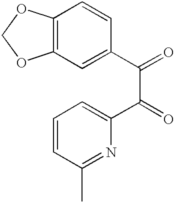

1-Benzo[1,3]dioxol-5-yl-2-(6-methyl-pyridin-2-yl)-ethanone (3 g, 1.7 mmol) (prepared according to the method described in U.S. Pat. No. 3,940,486) was dissolved in dimethyl sulfoxide (50 ml) and heated to 60° C. Hydrogen bromide (11.9 ml of a 48% solution in water) was added dropwise and the reaction stirred for 3 hours at 60° C. The cooled reaction was poured into water (100 ml) and the pH adjusted to pH 8 with saturated sodium bicarbonate solution. The organic product was extracted into ethyl acetate (3×100 ml), dried (MgSO₄) and evaporated to dryness under reduced pressure. The title compound was isolated by silica gel column chromatography using ethyl acetate as eluent (2.35 g, 74%). ¹H NMR (250 MHz, CDCl₃) δ:2.51 (3H, s), 6.08 (2H, s), 6.86 (1H, d), 7.37 (1H, d), 7.42 (1H, dd), 7.46 (1H, d), 7.78 (1H, dt), 7.97 (1H, d); m/z (API⁺): 270 (MH⁺).

### Description 2

### 1-(6-Methyl-pyridin-2-yl)-2-quinoxalin-6-yl-ethane-1,2-dione 1-oxime (D2)

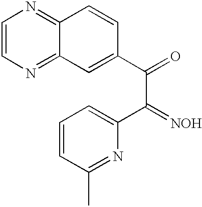

2-(6-Methyl-pyridin-2-yl)-1-quinoxalin-6-yl-ethanone (prepared according to the method described in U.S. Pat. No. 3,940,486) (3.3 g, 12.5 mmol) was dissolved in a 5M hydrogen chloride solution and treated with a sodium nitrite (10 g, 14.5 mmol) and water (10 ml) solution, whilst the reaction mixture was stirred vigorously. The reaction mixture was stirred at ambient temperature for one hour then quenched with ammonium chloride (40 ml) and the pH adjusted to pH8 with 2M sodium hydroxide solution. The organic product was extracted into ethyl acetate (2×100 ml), dried (MgSO₄) and evaporated to dryness under reduced pressure. The title compound was isolated by silica gel chromatography using an equal ratio of ethyl acetate to petroleum ether as an eluent, (3.1 g, 83%); m/z (API⁺): 293 (MH⁺).

### Description 3

### 1-(6-Methyl-pyridin-2-yl)-2-(4-methoxyphenyl)-ethane-1,2-dione (D3)

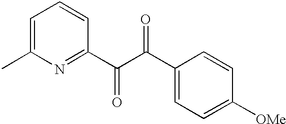

2-(6-Methyl-pyridin-2-yl)-1-(4-methoxyphenyl)-ethanone (1.7 g) (prepared according to the method described in U.S. Pat. No. 3,940,486) was dissolved in dimethyl sulfoxide (30 ml) and heated to 70° C. 48% aqueous HBr (7 ml) was added dropwise and heating continued for a further 3 h. On cooling, the mixture was poured onto ice, neutralised with solid sodium bicarbonate and extracted with ethyl acetate. The organic extracts were dried (MgSO₄) and concentrated in vacuo to afford the title compound as a yellow oil; m/z (API⁺): 256 (MH⁺).

### Description 4

### 2-Amino-5-[2-tert-butyl-5-(6-methylpyridin-2-yl)-1H-imidazol-4-yl]-phenol hydrochloride (D4)

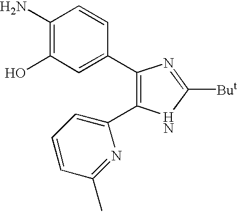

Example 71 (2 g, 6 mmol) was dissolved in 2M aqueous HCl (50 ml). After stirring at ambient temperature for 2 h the solution was concentrated in vacuo to afford the title compound as a yellow solid. m/z (API+) 325.

### Description 5

### N′-(5-Bromo-2-aminopyridine)-N,N-dimethylformamidine (D5)

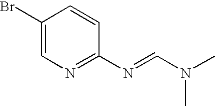

5-Bromo-2-aminopyridine (9.8 g, 56.6 mmol, 1 eq) was dissolved in dry DMF (20 ml) and dry dimethylformamide dimethylacetal (20 ml) under argon. The solution was refluxed at 130° C. for 16 h, allowed to cool, and the solvents removed. The resultant residue was used in the next stage without purification. m/z [APCIMS]: 228./230. [M+H]⁺.

### Description 6

### 6-Bromo-[1,2,4] triazolo[1,5-a] pyridine (D6)

D5 (16.2 g, ˜56.6 mmol, 1 eq) was dissolved in methanol (90 ml) and pyridine (10 ml) under

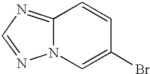

argon and cooled down to 0° C. To this was added, with stirring, hydroxylamine-O-sulfonic acid (7.3 g, 75.2 mmol, 1.3 eq) to form a purple suspension. This was allowed to reach room temperature and stirred for 16 h. After removing the solvents, the residue was suspended in aqueous sodium hydrogen carbonate (200 ml) and extracted with ethyl acetate (2×200 ml). The organic layer was then washed with water and brine (100 ml of each), dried (MgSO₄) and the solvent removed. Purification by flash chromatography on silica, eluting with a gradient solvent system of first 2:1 40-60° C. petroleum ether:ethyl acetate to 1:1 40-60° C. petroleum ether:ethyl acetate afforded the product as a pale yellow solid (5 g, 44.6%); ¹H NMR (250 MHz, CDCl₃) δ: 7.65 (1H, d), 7.69 (1H d), 8.34 (1H, s), 8.77 (1H, s),; m/z [APCIMS]: 198/200 [M+H]⁺.

### Description 7

### 6-Trimethylsilanylethynyl-[1,2,4] triazolo[1,5-a] pyridine (D7)

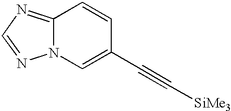

D6 (5 g, 25.26 mmol, 1 eq) was dissolved in THF (50 ml) and argon bubbled through the solution for five minutes. To this was added copper iodide (0.46 g, 2.53 mmol, 0.1 eq), dichlorobistriphenylphosphine palladium(0) (0.36 g, 0.51 mmol, 0.02 eq), and trimethylsilylacetylene (7.14 ml, 4.96 g, 50.52 mmol, 2 eq). Diisopropylamine (6.78 ml, 5.1 g, 50.52 mmol, 2 eq) was added dropwise to the solution and the resulting deep red suspension stirred under argon for 24 h. This was then filtered through celite, washing with an excess of ethyl acetate, and the solvents removed. The residue was then suspended in water (200 ml) and extracted with ethyl acetate (2×200 ml), and the organic layers combined, washed with water and brine (100 ml of each), dried (MgSO₄), and the solvent removed. Purification by flash chromatography over silica, eluting with 3:1 40-60° C. petroleum ether: ethyl acetate afforded the product as a pale yellow solid (2.9 g, 53.3%). ¹H NMR (400 MHz, CDCl₃) δ: 0.28 (9H, s), 7.54 (1H, d), 7.69 (1H, d), 8.36 (1H, s), 8.72 (1H, s); m/z [APCIMS]: 216 [M+H]⁺.

### Description 8

### 6-Ethynyl-[1,2,4]triazolo[1,5-a] pyridine (D8)

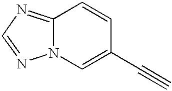

D7 (2.9 g, 13.47 mmol, 1 eq) was dissolved in methanol and to this was added potassium carbonate (5.6 g, 40.4 mmol, 3 eq). The suspension was stirred for 2 h and the solvent removed. The residue was suspended in water (100 ml) and extracted with ethyl acetate (2×100 ml). The organic layers were then combined, washed with water and brine (50 ml of each), dried (MgSO₄), and the solvent removed to give a pale orange solid (1.8 g, 95%) that was used in the next reaction without further purification. m/z [APCIMS]: 144.1 [M+H]⁺.

### Description 9

### 6-(6-Methylpyridin-2-ylethynyl)-[1,2,4] triazolo[1,5-a] pyridine (D9)

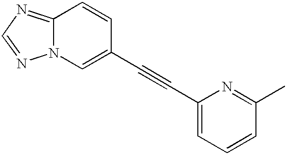

D8 (1.8 g, 12.56 mmol, 1 eq) was dissolved in anhydrous THF (50 ml) and TMEDA (50 ml) under argon. To this was added tetrakis(triphenylphosphine) palladium(0) (0.72 g, 0.63 mmol, 0.05 eq), copper iodide (0.24 g, 1.26 mmol, 0.1 eq) and 2-bromo-6-methylpyridine (4.32 g, 25.12 mmol, 2 eq). The mixture was then refluxed at 60° C. for 5 h, allowed to cool, and the solvents removed. The residue was suspended in ethyl acetate and water (100 ml of each) and filtered through celite, washing with more ethyl acetate (100 ml). The aqueous layer was washed with further ethyl acetate (50 ml) and the organic layers combined. The organic solution was washed with water and brine (100 ml of each), dried (MgSO₄) and the solvent removed. Purification by flash chromatography over silica, eluting with ethyl acetate, afforded the title compound as a pale yellow solid (1 g, 34%). ¹H NMR (400 MHz, CDCl₃) δ: 2.61 (3H, s), 7.18 (1H, d), 7.40 (1H, d), 7.63 (1H, t), 7.68 (1H, d), 7.76 (1H, d), 8.40 (1H, s), 8.86 (1H, s); m/z [APCIMS]: 235 [M+H]⁺.

### Description 10

### 1-(6Methylpyridin-2-yl)-2-[1,2,4]triazolo[1,5a]pyridin-6-yl-ethane-1,2-dione (D10)

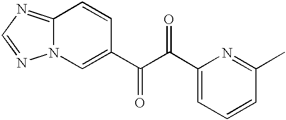

A mixture of the acetylene (0.200 g, 0.854 mmol, 1.0 eq) and palladium(II) chloride (0.015 g, 0.085 mmol, 0.1 eq) in dry DMSO (4 ml) was heated at 140° C. for 5 h then allowed to cool to room temperature. Water and ethyl acetate were added and the entire solution filtered through Kieselguhr. The layers were separated and the aqueous was extracted with more ethyl acetate. The combined organic phase was washed with water, brine and dried (MgSO₄). Concentration followed by column chromatography over silica, eluting with 50% Petrol-EtOAc-EtOAc afforded the title compound as a white solid, 0.090 g, 40%. ¹H NMR (400 MHz; CDCl₃) δ: 2.50 (3H, s), 7.41 (1H, d), 7.83 (1H, d), 7.88 (1H, d), 8.03 (1H, d), 8.13 (1H, d), 8.47 (1H, s), 9.11 (1H, s); m/z [ESMS]: 267.1 [M+H]⁺.

### Description 11

### 2-[2-tert-Butyl-5-(4-methoxy-3-nitrophenyl)-3H-imidazol-4-yl]-6-methylpyridine (D11)

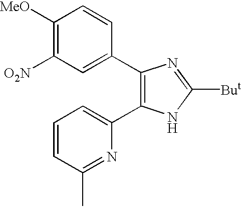

Example 17 (2.88 g, 9 mmol) was dissolved in dichloromethane (19 ml). Ammonium nitrate (1.15 g, 14.3 mmol) and trifluoroacetic anhydride (4.05 ml, 28.7 mmol) were added and the mixture heated at reflux for 5 h after which time more ammonium nitrate (575 mg, 7.1 mmol) and trifluoroacetic anhydride (2.20 ml, 14.3 mmol) were added. After a further 1 h reflux the reaction mixture was cooled, diluted with more dichloromethane and washed with aq. sodium bicarbonate, water and brine. The organic phase was dried over sodium sulfate and evaporated to dryness to afford the title compound (3.3 g). m/z [ESMS]: 367.2 [M+H]⁺.

### Description 12

### 2-[2-tert-Butyl-5-(4-hydroxy-3-nitrophenyl)-3H-imidazol-4-yl]-6-methylpyridine (D12)

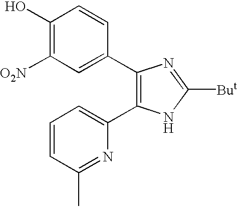

D11 (1.07 g, 2.9 mmol) was dissolved in dry DMF ( 15 ml). Lithium chloride (370 mg, 8.8 mmol) was added and the mixture heated at 160° C. under argon overnight. On cooling, all volatiles were removed in vacuo and the residue partitioned between aq. ammonium chloride and ethyl acetate. The organic phase was dried over sodium sulfate and concentrated in vacuo to afford the title compound (1.0 g). m/z [ESMS]: 353.2 [M+H]⁺.

### Description 13

### {4-[2-tert-Butyl-5-(6-methylpyridin-2-yl)-1H-imidazol-4-yl]-2-nitropnenoxy}-Acetic Acid Ethyl Ester

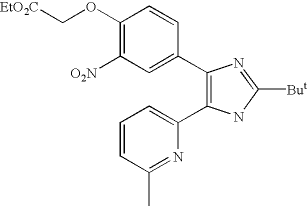

D12 (770 mg, 2.2 mmol) was dissolved in dry DMF (10 ml). Ethyl bromoacetate (486 ul, 4.4 mmol) and potassium carbonate (906 mg, 6.6 mmol) were added and the mixture stirred at 60° C. under argon overnight. On cooling, the reaction mixture was diluted with water and extracted with ethyl acetate. The organic phase was dried (MgSO₄), concentrated in vacuo and the residue subjected to column chromatography eluting with 2:1 ethyl acetate:hexane to afford the title compound (465 mg) m/z [ESMS]: 439.3 [M+H]⁺.

### Example 1

### 2-[5-Benzo[1,3]dioxol-5-yl-2-(1,1-dimethoxy-methyl)-3H-imidazol-4-yl]-6-methyl-pyridine

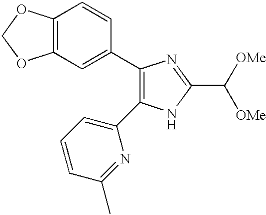

D1 (2 g, 7.4 mmol) was dissolved in tert-butyl methyl ether (20 ml) and treated with glyoxal 1,1-dimethyl acetal (2.6 ml of 45% solution in tert-butyl methyl ether). Ammonium acetate (1.49 g) in methanol (10 ml) was added and the reaction stirred at room temperature for 3 hours. The pH of the reaction was adjusted to pH 8 with saturated sodium carbonate solution. The reaction mixture was partitioned between dichloromethane (100 ml) and water (100 ml). The dichloromethane layer was separated, dried (MgSO₄) and evaporated to dryness under reduced pressure to yield the title compound (2.4 g, 91%). ¹H NMR (250 MHz, CDCl₃) δ: 2.53 (3H, s), 3.43 (6H, s), 5.53 (1H, s), 5.99 (2H, s), 6.84 (1H, d, J=8 Hz), 6.96 (1H, d, J=7 Hz), 7.10-7.13 (2H, m), 7.32 (1H, d, J=8 Hz), 7.45 (1H, t, J=8 Hz), NH not observed; m/z (API⁺): 354 (MH⁺).

### Example 2

### 4-Benzo[1,3]dioxol-5-yl-5-(6-methyl-pyridin-2-yl)-1H-imidazole-2-carboxylic Acid Ethyl Ester

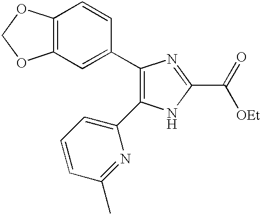

Prepared from D1 (0.3 g, 1.1 mmol) and ethyl glyoxylate (0.34 ml of a 50% solution in toluene) according to the procedure of Example 1. The title compound was isolated by silica gel column chromatography using a 1:9:190 ammonia: methanol:dichloromethane solution as eluent (0.089 g, 23%). ¹H NMR (250 MHz, CDCl₃) δ: 1.44 (3H, t, J=7 Hz), 2.58 (3H, s), 4.48 (2H, q, J=7 Hz), 6.01 (2H, s), 6.85 (1H, d, J=8 Hz), 7.01 (1H, d, J=8 Hz), 7.09-7.13 (2H, m), 7.33 (1H, d, J=8 Hz), 7.45 (1H, t, J=8 Hz), NH not observed; m/z (API⁺): 352 (MH⁺).

### Example 3

### 4-Benzo[1,3]dioxol-5-yl-5-(6methyl-pyridin-2-yl)1H-imidazole-2-carboxylic Acid Amide

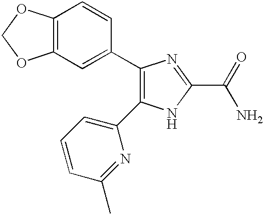

Example 2 (0.2 g, 0.57 mmol) was dissolved in methanol (50 ml). Ammonia gas was bubbled through the solution (15 min) until saturation. The reaction flask was stoppered and left to stand at room temperature for 7 days before solvent removal under reduced pressure. The title compound was isolated by silica gel column chromatography using ethyl acetate as eluent (0.053 g, 29%). ¹H NMR (250 MHz, CDCl₃) δ: 2.55 (3H, s), 5.85 (1H, brs), 6.02 (3H, m), 6.88 (1H, d), 7.00-7.12 (3H, m), 7.28 (1H, d), 7.47 (1H, t), 11.25 (1H, brs); m/z (API⁺): 323 (MH⁺).

### Example 4

### 5-[4-Benzo[1,3]dioxol-5-yl]-5-(6-methyl-pyridin-2-yl)-1H-imidazol-2-yl-pentanoic Acid Methyl Ester

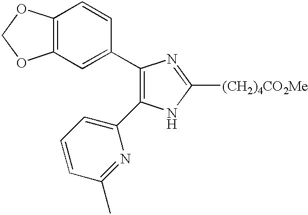

D1 (1.24 g, 4.6 mmol) was dissolved in tert-butyl methyl ether (50 ml) and treated with adipic semialdehyde methyl ester, (1 g, 6.9 mmol). Ammonium acetate (3.55 g) in methanol (50 ml) was added and the reaction heated at reflux temperature for 18 hours. Solvent was removed from the cooled reaction under reduced pressure and the residue partitioned between sodium hydroxide (50 ml of a 2 M solution in water) and dichloromethane (100 ml). The dichloromethane layer was separated, dried (MgSO₄) and evaporated to dryness under reduced pressure. The title compound was isolated by silica gel column chromatography using a 1:9:190 ammonia:methanol:dichloromethane solution as eluent (1.15 g, 63%). 1H NMR (250 MHz, CDCl₃) δ: 1.52-1.90 (4H, m), 2.30-2.40 (2H, m), 2.54 (3H, s), 2.80 (2H, brt, J=7 Hz), 3.67 (3H, s), 5.99 (2H, s), 6.84 (1H, d, J=9 Hz), 6.92(1H, d, J=8 Hz), 7.08 (1H, s), 7.11 (1H, d, J=8 Hz), 7.29 (1H, d, J=8 Hz), 7.40 (1H, t, J=8 Hz), 10.17 (1H, brs); m/z (API⁺): 394 (MH⁺).

### Example 5

### 5-[4-Benzo[1,3]dioxol-5-yl-5-(6-methyl-pyridin-2-yl)-1H-imidazol-2-yl-pentanoic Acid Amide

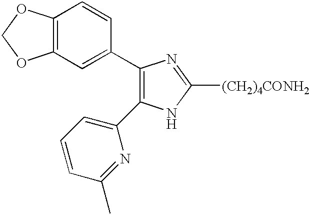

Prepared from Example 4 (1 g, 25 mmol) using the procedure of Example 3. 5-[4-Benzo[1,3]dioxol-5-yl-5-(6-methyl-pyridin-2-yl)-1H-imidazol-2-yl]-pentanoic acid amide was isolated by silica gel column chromatography using a 1:9:190 ammonia:methanol:dichloromethane solution as eluent (0.32 g, 33%). 1H NMR (250 MHz, CDCl₃) δ: 1.55-1.73 (4H, m), 2.19 (2H, t, J=7 Hz), 2.46 (3H, s), 2.76 (2H, t, J=7 Hz), 5.46 (1H, brs), 5.99 (2H, s), 6.32 (1H, brs), 6.83 (1H, d, J=8 Hz), 6.95 (1H, d, J=7 Hz), 7.07 (1H, s), 7.09 (1H, d, J=8 Hz), 7.30 (1H, d, J=8 Hz), 7.43 (1H, t, J=8 Hz), NH not observed; m/z (API⁺): 379.

### Example 6

### 4-Benzo[1,3]dioxol-5yl-5-(6-methyl-pyridin-2-yl)-1H-imidazole-2 Carboxaldehyde

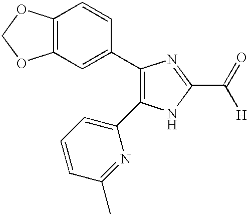

Example 1 (0.3g, 0.85 mmol) was dissolved in hydrochloric acid (20 ml of a 2M solution in water) and heated at reflux temperature for 3 hours. The cooled solution was neutralised with saturated sodium bicarbonate and the product extracted into dichloromethane. The dichloromethane solution was dried (MgSO₄) and the title compound isolated by solvent evaporation under reduced pressure (0.22 g, 84%). 1H NMR (250 MHz CDCl₃) δ: 2.53 (3H, s), 6.03 (2H, brs), 6.89 (1H, d, J=8 Hz), 7.03-7.15 (4H, m), 7.37 (1H, d, J=8 Hz), 7.50 (1H, t, J=8 Hz), 9.76 (1H, s); m/z (API⁺): 308 (MH⁺).

### Example 7

### 3-[4Benzo[1,3]dioxol-5-yl-5-(6-methyl-pyridin-2-yl)-1H-imidazol-2-yl]-acrylonitrile

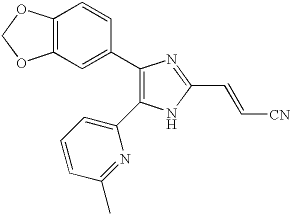

Example 6 (0.76 g, 2.47 mmol) was dissolved in dichloromethane (100 ml). Cyanomethyl triphenyl phosphonium chloride (0.826 g, 2.47 mmol) was added followed by diisopropyl ethylamine (0.85 ml, 48.7 mmol). The reaction mixture was stirred for 3 hours at room temperature then partitioned between water (200 ml) and dichloromethane (100 ml). The dichloromethane layer was separated, dried (MgSO₄) and evaporated to dryness under reduced pressure. 3-[4-Benzo[1,3]dioxo-5-yl-5-(6-methyl-pyridin-2-yl)-1H-imidazol-2-yl]acrylonitrile was isolated by silica gel column chromatography using a 1:9:190 ammonia:methanol:dichloromethane solution as eluent (0.33 g, 41%). m/z (API⁺): 331 (MH⁺).

### Example 8

### (E)-3-[4-Benzo[1,3]dioxol-5-yl-5-(6-methyl-pyridin-2-yl)-1H-imidazol-2-yl]-acrylamide

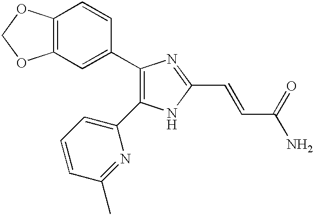

Example 7 (0.22 g, 0.67 mmol) was dissolved in tert-butanol (50 ml) and treated with potassium hydroxide (0.112 g, 2 mmol). The reaction mixture was heated at reflux temperature for 18 hours before solvent removal under reduced pressure. The title compound was isolated by isolated by silica gel column chromatography using ethyl acetate as eluent (0.03 g, 13%). ¹H NMR (250 MHz, CDCl₃) δ: 2.60 (3H, s), 5.68 (1H, brs), 5.90 (1H, d, J=13 Hz), 5.99 (2H, s), 6.29 (1H, brs), 6.83 (1H, d, J=8 Hz), 6.93 (1H, d, J=13 Hz), 6.97 (1H, d, J=8 Hz), 7.12 (1H, d, J=8 Hz), 7.33 (1H, d, J=8 Hz), 7.40-7.72 (3H, m); m/z (API⁺): 349 (MH⁺).

### Example 9

### 2-(5-Benzo(1,3]dioxol-5-yl-2-tert-butyl-3H-imidazol-4-yl)-6-methylpyridine

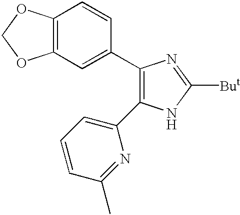

The title compound (280 mg, 83%) was prepared from DI (269 mg, 1 mmol) and pivalaldehyde (129 mg, 1.5 mmol), as described in Example 4, and isolated as a white foam, after chromatography on silica gel using ethyl acetate in 60-80° petroleum ether as eluent: 1H NMR (hydrochloride salt, 250 MHz, CD₃OD) δ: 1.32 (9H, s), 2.48 (3H, s), 5.79 (2H, s), 6.68-6.78 (3H, m), 7.19 (1H, d, J=8Hz), 7.33 (2H, d, J=8Hz), 7.75 (1H, t, J=8Hz); m/z (API⁺): 336 (MH⁺).

### Example 10

### 6-[2-Ethyl-5-(6-methyl-pyridin-2-yl)-1H-imidazol-4yl]-quinoxaline

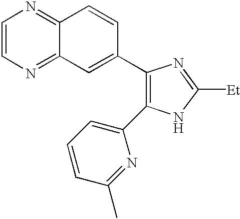

D2 (5 g, 1.71 mmol) was dissolved in acetic acid (50 ml) and treated with ammonium acetate (2.64 g, 34.3 mmol) and propionaldehyde (0.12 ml, 1.71 mmol) and heated at 100° C. for 30 minutes. The pH of the cooled reaction mixture was adjusted to pH8 at 0° C. with a 2M sodium hydroxide solution. Organic product was extracted into dichloromethane (2×100 ml), dried (MgSO₄) and evaporated to dryness under reduced pressure, m/z (API⁺): 332 (MH⁺). Crude 2-ethyl-5-(6-methyl-pyridin-2-yl)-4-quinoxalin-6-yl-imidazol-1-ol (518 mg, 1.56 mmol) was dissolved in DMF, treated with triethylphosphite (0.83 ml, 4.68 mmol) and stirred at 130° C. for five hours. The DMF was removed under reduced pressure and the product was partitioned between ethyl acetate (100 ml) and water (100 ml). Organic product was dried (MgSO₄) and evaporated to dryness under reduced pressure. The title compound was purified by silica gel column chromatography eluting with 5% methanol in dichloromethane (300 mg, 56%); 1H NMR (250 MHz, CDCl₃) δ: 1.42 (3H, t, J=7.5 Hz), 2.56 (3H, s), 2.89 (2H, q; J=7.5 Hz), 6.99 (1H, d, J=7.5Hz), 7.39-7.48 (2H, m), 8.12 (2H, s), 8.40 (1H, s), 8.82-8.85 (2H, m), NH not observed; m/z (API⁺): 316 (MH⁺).

### Example 11

### 6-[2-Ethyl-3-methyl-5-(6-methyl-pyridin-2-yl)-3H-imidazol-4-yl]-quinoxaline

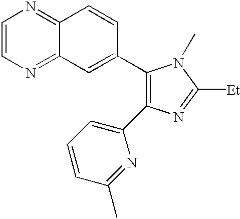

Example 10 (100 mg, 0.32 mmol) was dissolved in dry tetrahydrofuran (50 ml), cooled to 0° C. and treated with sodium bis(trimethylsilyl)amide (0.35 ml, 0.35 mmol) and stirred at this temperature for 15 min before the addition of iodomethane (30 μl, 0.48 mmol). The reaction mixture was stirred at an ambient temperature for one hour, then product was diluted with water and extracted into dichloromethane (2×100 ml). The organic product was dried (MgSO₄) and evaporated to dryness under reduced pressure (55 mg, 52%); 1H NMR (250 MHz, CDCl3) δ: 1.26-1.29 (3H, m), 2.15 (3H, s), 7.73 (2H, q, J=7.5 Hz), 3.38 (3H, s), 6.74 (1H, d, J=7.5Hz), 7.17-7.28 (2H, m), 7.63-7.68 (1H, m), 7.92-7.97 (2H, m), 8.72 (2H, s); m/z (API⁺): 330 (MH⁺).

### Example 12

### 6-[2-Isopropyl-5-(6-methyl-pyridin-2-yl)-1H-imidazol-4-yl]-quinoxaline

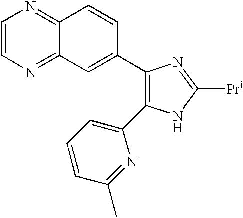

Prepared from D2 and isobutyraldehyde according to the procedure of Example 10. ¹H NMR (250 MHz, CDCl₃) δ: 1.38-1.41 (6H, m), 2.50 (3H, s), 3.18 (1H, m), 7.35 (1H, d, J=7.5 Hz), 7.30-7.45 (2H, m), 8.13 (2H, s), 8.40 (1H, s), 8.81-8.84 (2H, m), NH not observed; m/z (API⁺): 330 (MH⁺).

### Example 13

### 6-[2-Isopropyl-3-methyl-5-(6-methyl-pyridin-2-yl)-3H-imidazol-4-yl]-quinoxaline

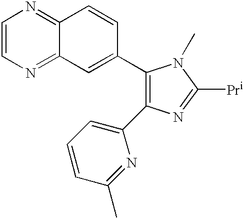

Prepared from Example 12 according to the procedure of Example 11. ¹H NMR (250 MHz, CDCl₃) δ: 1.31 (6H, d, J=7.5 Hz), 2.12 (3H, s), 3.42 (3H, s), 3.02 (1H, m), 6.74 (1H, t, J=5 Hz), 7.28-7.29 (2H, m), 7.65-7.69 (1H, m), 7.92-7.98 (2H, m), 8.73 (2H, s); m/z (API⁺): 334 (MH⁺).

### Example 14

### 6-2-Methyl-5-(6-methyl-pyridin-2-yl)-1H-imidazol-4-quinoxaline

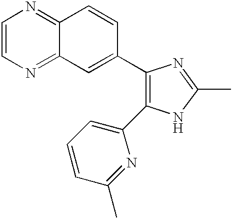

Prepared from D2 and acetaldehyde according to the procedure of Example 10. ¹H NMR (250 MHz, CDCl₃) δ:2.67 (3H, s), 2.81 (3H, s), 7.49 (2H, t, J=8.0 Hz), 7.86-8.00 (2H, m), 8,24 (1H, d, J=8.75 Hz), 8.37 (1H, s), 8.99 (2H, s), NH not observed; m/z (API⁺): 302 ( MH⁺).

### Example 15

### 6-[2,3-Dimethyl-5-(6-methyl-pyridin-2-yl)-3H-imidazol-4-yl]-quinoxaline

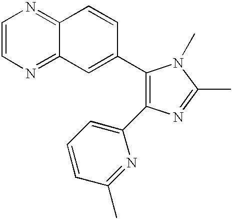

Prepared from Example 14 according to the procedure of Example 11. ¹H NMR (250 MHz, CDCl₃) δ: 2.32 (3H, s), 2.57 (3H, s), 3.52 (3H, s), 6.89 (1H, d, J=7.5 Hz), 7.28 (1H, s), 7.36-7.45 (1H, m), 7.79-7.83 (1H, m), 8.11 (2H, d, J=10 Hz), 8.89 (2H, s); m/z (API⁺): 316 ( MH⁺).

### Example 16

### 6-[2-tert-Butyl-5-(6-methyl-pyridin-2-yl)-1H-imidazol-4-yl]-quinoxaline

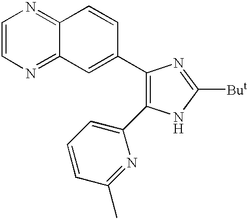

Prepared from D2 and pivalaldehyde according to the procedure of Example 10. ¹H NMR (250 MHz; CDCl₃) δ: 1.43 (9H, s), 2.78 (3H, s), 6.97 (1H, d, J=7.5 Hz), 7.31 (1H, s), 7.42 (1H, t, J=7.5 Hz), 8.09-8.18 (2H, m), 8.40 (1H, s), 8.82-8.87 (2H, m), NH not observed; m/z [ESMS]: 344.2 [M+H]⁺

### Example 17

### 2-[tert-Butyl-5-(4-methoxyphenyl)-3H-imidazol-4-yl]-6-methylpyridine

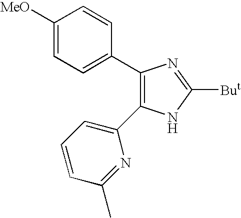

Prepared from D3 and pivalaldehyde according to the procedure of Example 4. ¹H NMR (250 MHz, CDCl₃) δ: 1.41 (9H, s), 2.42 (3H, s), 3.84 (3H, s), 6.91 (3H, m), 7.17 (1H, d), 7.42 (1H, t), 7.51 (2H, m), NH not observed; m/z (API⁺) 322 (MH⁺).

### Example 18

### 2[Methyl-5-(4-methoxyphenyl)-3H-imidazol-4-yl]-6-methylpyridine

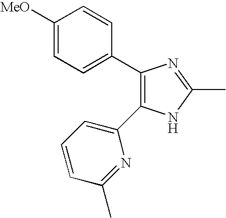

D3 (250 mg, 0.1 mmol) was dissolved in tert-butyl methylether (20 ml) and methanol (5 ml). Acetaldehyde (2 ml) was added and the mixture heated at reflux overnight. Further portions of acetaldehyde (3×1 ml) were added at 2, 4 and 6 h. On cooling the reaction mixture was diluted with ethyl acetate and washed sequentially with aq. sodium bicarbonate, water and brine. The organic phase was dried (Na₂SO₄) and concentrated in vacuo to afford a brown oil which was subjected to dry flash chromatography on silica gel eluting with 5% methanol in dichloromethane to afford a pale yellow oil. ¹H NMR (250 MHz, CDCl₃) δ: 2.43 (3H, s), 2.51 (3H, s), 3.84 (3H, s), 6.92 (3H, m), 7.27 (1H, d), 7.38 (1H, t), 7.52 (2H, m), NH not observed; m/z (API⁺) 322 (MH⁺).

### Example 19

### 7-[2-tert-Butyl-5-(6-methylpyridin-2-yl)-1H-imidazol-4-yl]-4H-benzo[1,4]oxazin-3-one

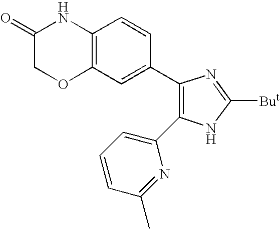

To a solution of D4 (30 mg, 0.084 mmol, 1.0 eq) in dry DMF (0.5 ml) under argon at room temperature was added chloroacetyl chloride (10 mg, 0.092 mmol, 7.5 μl, 1.1 eq). Potassium carbonate (46 mg, 0.334 mmol, 4.0 eq) was added portionwise and the resultant mixture stirred for 16 h at room temperature. The reaction mixture was diluted with water (10 ml) and extracted with EtOAc (2×10 ml). The organic solution was washed with water and brine (20 ml of each) then dried (MgSO₄) and the solvents removed. Purification by flash column chromatography over silica, eluting with 9:1 CH₂Cl₂:MeOH +1% Et₃N afforded the title compound as an off white solid. ¹H NMR (400 MHz; DMSO-d⁶) δ: 1.52 (9H, s), 2.67 (3H, s), 4.63 (2H, s), 6.98 (1H, d), 7.11 (1H, d), 7.22 (1H, s), 7.28 (1H, d), 7.37 (1H, d), 7.80 (1H, t), 10.98 (1H, br.s), NH not observed; m/z [ESMS]: 363.2 [M+H]⁺.

### Example 20

### 6-[2-tert-Butyl-5(6-methylpyridin-2-yl)-1H-imidazol-4-yl]-3H-benzoxazol-2-one

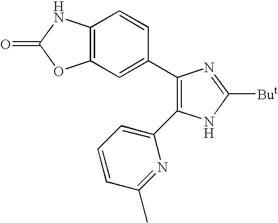

To a stirred solution of D4 (40 mg, 0.111 mmol, 1.0 eq) and 1,1′-carbonyldiimidazole (20 mg, 0.123 mmol, 1.1 eq) in anhydrous DMF (1.1 ml) under argon at room temperature was added dropwise triethylamine (56 mg, 77 μl, 5.0 eq). The resultant mixture was stirred at room temperature for 16 h then diluted with water (10 ml). The mixture was extracted with EtOAc (2×10 ml) and the organic solution washed with water and brine (20 ml of each) then dried (MgSO₄) and the solvents removed. Purification by flash column chromatography over silica, eluting with 25:1 CH₂Cl₂: MeOH+1% Et₃N afforded the title compound as an off white solid. ¹H NMR (250 MHz; CD₃OD) δ: 1.34 (9H, s), 2.41 (3H, s), 6.94 (11, d), 7.11-7.07 (2H, m), 7.14 (1H, d), 7.18 (1H, s), 7.46 (1H, t), NHs not observed; m/z [ESMS]: 349.2 [M+H]⁺.

### Example 21

### 7-[2-tert-Butyl-5-(6-methylpyridin-2-yl)1H-imidazol-4yl]-3,4-dihydro-2H-benzo[1,4]oxazine

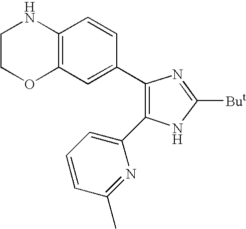

To a solution of Example 19 (19 mg, 0.052 mmol, 1.0 eq) in anhydrous THF (0.75 ml) under argon at room temperature was added dropwise LiAlH₄ solution (262 μ1 1M solution in ether, 0.262 mmol, 5.0 eq). An effervescence was observed as hydrogen was evolved and the resultant orange mixture was stirred at room temperature for 5 h. Methanol was added (1 ml) and the reaction mixture stirred vigorously with saturated aqueous potassium sodium tartrate solution (30 ml) and EtOAc (30 ml) for 2 h. The layers were separated and the organic washed with water, and brine (30 ml of each) and dried (MgSO₄) and the solvents removed. Purification by flash column chromatography over silica, eluting with 9:1 CH₂Cl₂:MeOH+1% Et₃N afforded the title compound as an off white solid. ¹H NMR (250 MHz; CD₃OD) δ: 1.33 (9H, s), 2.44 (3H, s), 3.24 (2H. t), 4.07 (2H, t), 6.48 (1H, d), 6.68-6.64 (2H, m), 6.99 (1H, d), 7.09 (1H, d), 7.44 (1H, t), NHs not observed; m/z [ESMS]: 349.3 [M+H]⁺.

### Example 22

### 2-[4-Benzo[1,3]dioxol-5-yl-5-(6-methylpyridin-2-yl)-1H-imidazol-2-yl]-methylamine

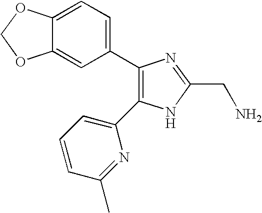

2-[4-Benzo[1,3]dioxol-5-yl-5-(6-methylpyridin-2-yl)-H-imidazol-2-yl-methyl]-isoindole-1,3-dione (3g ), prepared from D1 and (1,3-dioxo-1,3-dihydro-isoindol-2-yl)-acetaldehyde according to the procedure of Example 4, was dissolved in ethanol (200ml) and treated with hydrazine monohydrate (2 ml). The reaction was heated at reflux for 4 h, cooled, treated with acetone to quench excess hydrazone, and evaporated to dryness. The residue was then taken up in 2M hydrochloric acid, neutralised to pH 8 and extracted with dichloromethane. The combined organic layers were dried (MgSO₄), concentrated in vacuo and the residue subjected to dry flash chromatography on silica gel eluting with 90:9:1 dichloromethane, methanol, 0.88 ammonia to afford the title compound as an off white solid. ¹H NMR (250 MHz, CDCl₃) δ: 2.53 (3H, s), 4.05 (2H, s), 5.99 (2H, s), 6.83 (1H, d, J 6Hz), 6.94 (1H, d, J=7 Hz), 7.08 (2H, m), 7.28 (1H, d, J=10 Hz), 7.41 (1H, d, J=7Hz)NHs not observed; m/z (API+) 309.

### Examples 23-70

Stock solutions of 1-hydroxybenzotriazole (700 mg in 35 ml) and Example 22 (1.078 g in 35 mml) were made up in DMF. Excess N-cyclohexylcarbodiimide, N-methyl polystyrene was added to a Robbins FlexChem reaction block via a shallow 96 well plate. 1-Hydroxybenzotriazole solution (3 ml, 0.075 mmol) was added to to each well followed by the solution of Example 22 (0.5 ml, 0.05 mmol). Acids (0.1 mmol in 0.5 ml DMF) were then added to individual wells, the block sealed and shaken for 60 h. Resin bound isocyanate was then added and shaking continued for 18 h followed by addition of Amberlyte IRA-93 and a further 18 h shaking. Individual wells were then filtered and concentrated in vacuo to afford the coupled products.

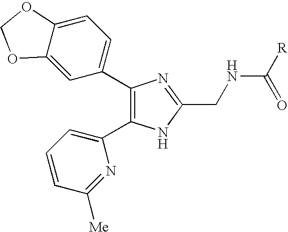

| | | m/z |
|---|---|---|
| Example | R | (API+) |
| 23 | 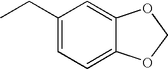 | 471 |
| 24 | 4-methoxybenzyl | 456 |
| 25 | 4-dimethylaminobenzyl | 471 |
| 26 | n-propyl | 379 |
| 27 | n-heptyl | 436 |
| 28 | 4-nitrobenzyl | 472 |
| 29 | cinnamyl | 439 |
| 30 | 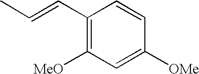 | 500 |
| 31 | —CH₂OPh | 443 |
| 32 | cyclohexyl | 419 |
| | —(CH₂)₃—Ph | 456 |
| 34 | benzyl | 427 |
| 35 | 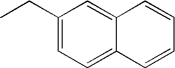 | 478 |
| 36 | 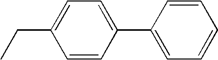 | 504 |
| 37 | 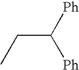 | 518 |
| 38 | 3-chlorobenzyl | 462 |
| 39 | 4-fluorobenzyl | 445 |
| 40 | 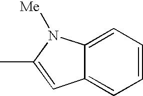 | 467 |
| 41 | 4-phenoxyphenyl | 506 |
| 42 | 4-benzoylphenyl | 518 |
| 43 | 4-acetylphenyl | 455 |
| 44 | 3-nitrophenyl | 458 |
| 45 | 4-nitrophenyl | 458 |
| 46 | 3,5-dichlorophenyl | 482 |
| 47 | 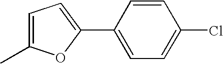 | 514 |
| 48 | 3-thiophenyl | 419 |
| 49 | 2-methoxy-4- | 490 |
| | thiomethylphenyl | |
| 50 | 6-methyl-pyridin-3-yl | 428 |
| 51 | 6-chloro-pyridin-3-yl | 449 |
| 52 | 2,6-dimethoxy | 474 |
| | pyridin-3-yl | |
| 53 | 2-naphthyl | 464 |
| 54 | 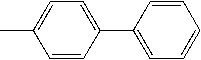 | 490 |
| 55 | 3-bromophenyl | 492 |
| 56 | 2-quinolyl | 464 |
| 57 | 2-pyrazinyl | 415 |
| 58 | 4-pyridyl | 414 |
| 59 | 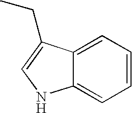 | 466 |
| 60 | 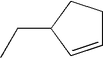 | 417 |
| 61 | 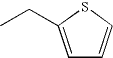 | 433 |
| 62 | 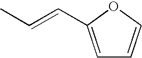 | 429 |
| 63 | —(CH₂)₂—C(O)Ph | 469 |
| 64 | 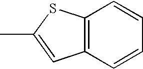 | 469 |
| 65 | —CH₂SPh | 460 |
| 66 | 4-methoxyphenyl | 443 |
| 67 | benzofuran-2-yl | 453 |
| 68 | 4-trifluomethylphenyl | 481 |
| 69 | piperonyl | 457 |
| 70 | 4-n-pentyloxyphenyl | 500 |

### Example 71

### 6-[2-tert-Butyl-5-(6-methyl-pyridin-2-yl)-1H-imidazol-4-yl]-benzoxazole

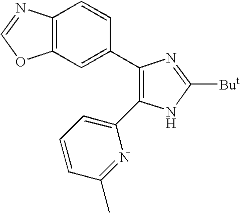

Prepared from 1-benzoxalol-6-yl-2-(6-methylpyridin-2-yl)-ethane-1,2-dione 2-oxime (prepared via the oximinoketone route described in Scheme 1) and pivalaldehyde according to the method of Example 10. ¹H NMR(250 MD, CDCl₃) δ: 1.40 (9H, s), 2.40 (3H, s), 6.94 (1H, d, J=8 Hz), 7.19 (1H, d, J=8 Hz), 7.62 (1H, t, J=8 Hz), 7.65 (1H, dd, J=8 and 1Hz), 7.89 (1H, s), 8.10 (1H, s), 11.06 (1H, br.s), NH not observed; m/z [API]: 333.1 [M+H]⁺

### Example 72

### 6-[2-tert-Butyl-5-(6-methylpyridin-2-yl)-1H-imidazol-4-yl]-[1,2,4]triazolo[1,5-a]pyridine

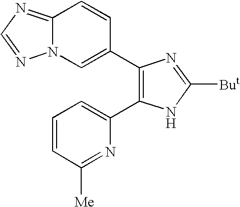

Prepared from 1-(6-methylpyridin-2-yl)-2-[1,2,4]triazolo[1,5a]pyridin-6-yl-ethane-1,2-dione (D10) and pivaldehyde according to the method of Example 4. ¹H NMR (250 MHz; CDCl₃)δ: 1.36 (9H, s), 2.35 (3H, s), 7.02 (1H, d), 7.17 (1H, d), 7.51 (1H, t), 7.78 (2H, s), 8.38 (1H, s), 8.91 (1H, s), NH not observed; m/z [CIMS]: 333 [M+H]⁺.

### Example 73

### 6-[2-tert-Butyl-5-(6-methylpyridin-2-yl)-1H-imidazol-4yl]-1H-benzimidazole

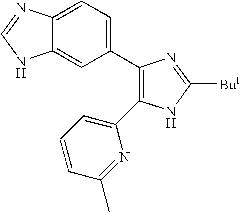

To a stirred solution of a mixture of 1- and 3-benzyl-5-[2-tert-butyl-5-(6-methylpyridin-2-yl)-1H-benzimidazole prepared via the diketone route described in Scheme 1) (1.53 g, 3.63 mmol, 1.0 eq) in anhydrous 1,4-dioxane (70 ml) under argon at room temperature was added dropwise a solution of sodium naphthalenide (91 ml 0.4M in THF, 36.3 mmol, 10.0 eq). The resultant brown mixture was stirred for a further 16 h under argon then open to the air for 20 min before partitioning between water and ethyl acetate. The organic phase was washed with water, brine, dried (MgSO₄) and concentrated to a yellow solid. The solid was triturated with 40-60 petrol to remove most of the naphthalene then purified by flash column chromatography, eluting with EtOAc→20% MeOH-EtOAc. The title compound was obtained as a yellow solid (0.780 g, 65%). ¹H NMR (400 MHz; CDCl₃) δ: 1.49 (9H, s), 2.52 (3H, s), 6.90 (1H, d), 7.23 (1H, d), 7.32 (1H, t), 7.41 (1H, d), 7.62 (1br.s), 7.87 (1H, s), 7.98 (1, br.s), NHs not observed; m/z [ESMS]: 332.2 [M+H]⁺.

### Example 74

### 6-[2-Isopropyl-5-(6-methypyridin-2-yl)-1H-imidazol-4-yl]-[1, 2,4]-triazolo-[1,5-a]pyridine

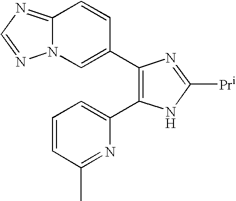

Prepared from D10 and isobutyraldehyde according to the method of Example 4. ¹H NMR(250 MHz; CDCl₃) δ: 1.31 (6H, d), 2.42 (3H, s), 3.12 (1H, h), 7.01 (1H, d), 7.22 (1H, d), 7.49 (1H, t), 7.76 (1H, d), 7.81 (1H, d), 8.36 (1H, s), 8.91 (1H, s), NH not observed; m/z [ESMS]: 319 [M+H]⁺.

### Example 75

### 5-[2-tert-Butyl-5-(6-methylpyridin-2-yl)-1H-imidazol-4-yl]-benzo[1,2,5]oxadiazole

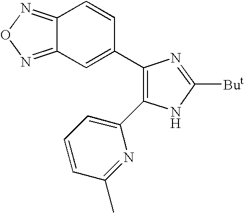

Prepared from 1-benzo[1,2,5]oxadiazol-5-yl-2-(6-methylpyridin-2-yl)-ethane-1,2-dione 2-oxime (prepared according to the route outlined in Scheme 1) and pivalaldehyde according to the method of Example 10. ¹H NMR (250 MHz, CDCl₃)δ: 1.59 (9H, s), 2.52 (3H, s), 7.02 (1H, d), 7.27 (1H, d), 7.48 (1H, t), 7.76 (1H, dd), 7.82 (1H, dd), 8.11 (1H, t), NH not observed; m/z [APCIMS]: 334.2 [M+H]⁺, 332.1 [M−H]⁻.

### Example 76

### 5-[2-Methyl-5-(6-methylpyridin-2-yl)-1H-imidazol-4-yl]-benzo[1,2,5]oxadiazole

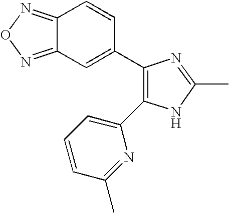

Prepared from 1-benzo[1,2,5]oxadiazol-5-yl-2-(6-methylpyridin-2-yl)-ethane-1,2-dione 2-oxime (prepared according to the route outlined in Scheme 1) and acetaldehyde according to the method of Example 10. ¹H NMR (250 MHz, CDCl₃) δ: 2.54 (3H, s), 2.58 (3H, s), 7.04 (1H, d), 7.30 (1H, d), 7.49 (1H, t), 7.76 (1H, dd), 7.83 (1H, dd), 8.11 (1H, s), NH not observed; m/z [APCIMS]: 292.1 [M+H]⁺, 290.1 [M−H]⁻.

### Example 77

### 5-[2-Isopropyl-5-(6-methylpyridin-2-yl)-1H-imidazol-4-yl]-benzo[1,2,5]oxadiazole

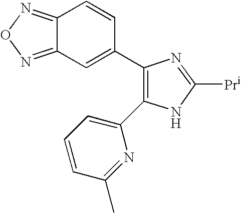

Prepared from 1-benzo[1,2,5]oxadiazol-5-yl-2-(6-methylpyridin-2-yl)-ethane-1,2-dione 2-oxime (prepared according to the route outlined in Scheme 1) and isobutyraldehyde according to the method of Example 10. ¹H NMR (250 MHz, CDCl₃) δ: 1.40 (6H, s), 2.54 (3H, s), 3.12 (1H, h) 7.04 (1H, d), 7.28 (1H, d), 7.49 (1H, t), 7.76 (1H, dd), 7.83 (1H, dd), 8.11 (1H, t), NH not observed; m/z [APCIMS]: 320.2 [M+H]⁺, 318.1 [M−H]⁻.

### Example 78

### 2-[2-tert-Butyl-5-(2,3-dihydrobenzofuran-5-yl)-3H-imidazol-4-yl]-6-methylpyridine

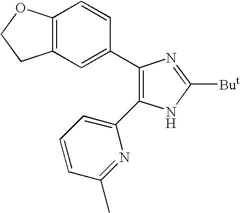

Prepared from 1-(2,3-dihydrobenzofuran-5-yl)-2-(6-methylpyridin-2-yl)-ethane-1,2-dione (prepared according to the route outlined in Scheme 1) and pivalaldehyde according to the method of Example 4. ¹H NMR (400 MHz, CDCl₃) δ: 1.43 (9H, s), 2.48 (3H, s), 3.22 (2H, t), 4.60 (2H, t), 6.77 (1H, d), 6.88 (1H, d), 7.24 (1H, d), 7.33 (2H, m), 7.48 (1H, s), NH not observed; m/z [APCIMS]: 334.3 [M+H]⁺, 332.2 [M−H]⁻.

### Example 79

### 5-[2-Ethyl-5-(6-methyl-pyridin-2-yl)-1H-imidazol-4-yl]-benzothiazole

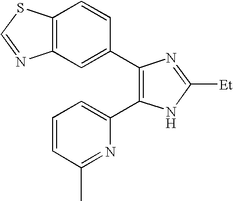

Prepared from 1-benzothiazol-5-yl-2-(6-methylpyridin-2-yl)-ethane-1,2-dione 2-oxime (prepared according to the route outlined in Scheme 1) according to the method of Example 10. ¹H NMR (250 MHz, CDCl₃) δ: 1.34 (3H, t), 2.51 (3H, s), 2.83 (2H, q), 6.98 (1H, d), 7.24-7.40 (2H, m), 7.77 (1H, dd), 7.99 (1H, d), 8.38 (1H, d), 9.01 (1H, s), NH not observed; m/z (API⁺): 321.1 (MH⁺).

### Example 80

### 5-[2-tert-Butyl-5-(6-methyl-pyridin-2-yl )-1H-imidazol-4-yl]-benzo[1,2,5]thiadiazole

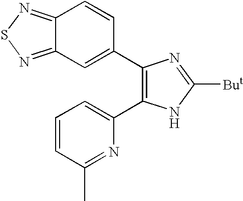

Prepared from 1-benzo[1,2,5]thiadiazol-5-yl-2-(6-methylpyridine-2-yl)-ethane-1,2-dione oxime (prepared according to the route outlined in Scheme 1) and pivalaldehyde according to the method of Example 4. ¹H NMR (250 MHz, CDCl₃) δ:1 .21(9H, s), 2.24 (3H, s), 6.91 (1H, d), 7.21 (1H, d), 7.39 (1H, t), 7.85-7.90 (2H, m), 8.20 (1H, s), 11.80 (1H, br. s); m/z (API⁺): 350.2 (MH⁺).

### Example 81

### 6-[2-tert-Butyl-5-(6-methyl-pyridin-2-yl)-1H-imidazol-4-yl]-benzothiazole

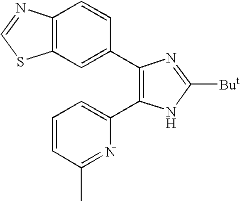

Prepared from 1-benzothiazol-5-yl-2-(6-methylpyridin-2-yl)-ethane-1,2-dione 2-oxime (prepared according to the route outlined in Scheme 1) and pivalaldehyde according to the method of Example 10. ¹H NMR (250 MHz, CDCl₃) δ:1.39 (9H, s), 2.38 (3H, s), 6.94 (1H, d, J=7.5 Hz), 7.20 (1H, d, J=7.5 Hz), 7.40 (1H, t, J=7.5 Hz), 7.75 (1H, dd, J=8.5 and 1.5 Hz), 8.10 (1H, d, J=8.5 Hz), 8.30 (1H, d, J=1.5 Hz), 9.00 (1H, s), 11.29 (1, br.s); m/z (API⁺): 349.2 (MH⁺).

### Example 82

### 6-[2-Methyl-5-(6-methyl-pyridin-2-yl)-1H-imidazol-4-yl]-benzothiazole

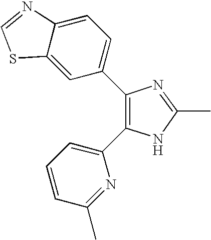

Prepared from 1-benzothiazol-5-yl-2-(6-methylpyridin-2-yl)-ethane-1,2-dione 2-oxime (prepared according to the route outlined in Scheme 1) and acetaldehyde according to the method of Example 10. ¹H NMR (250 MHz, CDCl₃) δ:2.50 (3H, s), 2.54 (3H, s), 6.97 (1H, d), 7.25-7.28 (1H, m), 7.40 (1H, t), 7.77 (1H, dd), 8.12 (1H, d), 8.27 (1H, d), 9.01 (1H, s), NH not observed; m/z (API⁺): 307.1 (MH⁺).

### Example 83

### 5-[2-Isopropyl-5-(6-methyl-pyridin-2-yl)-1H-imidazol-4-yl]-benzo[1,2,5]thiadiazole

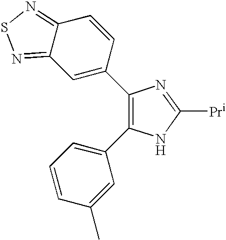

Prepared from 1-benzo[1,2,5]thiadiazol-5-yl-2-(6-methylpyridine-2-yl)-ethane-1,2-dione 2-oxime (prepared according to the route outlined in Scheme 1) and isobutyraldehyde. ¹H NMR (250 MHz, CDCl₃) δ: 1.29 (6H, d), 2.37 (3H, s), 3.06-3.23 (1H, m), 7.00 (1H, d), 7.31 (1H, d,), 7.47 (1H, t), 7.92-8.04 (2H, m), 8.27 (1H, s), 11.89 (1H, br.s); m/z (API⁺): 335.43 (MH⁺).

### Example 84

### 6-[2-Methyl-5-(6-methyl-pyridin-2-yl)-1H-imidazol-4-yl]-benzo[2,3]thiadiazole

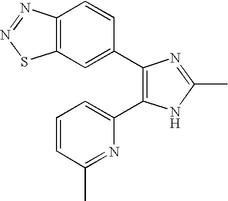

Prepared from 1-benzo[1,2,3]thiadiazol-6-yl-2-(6-methyl-pyridin-2-yl)-ethane-1,2-dione 2-oxime (prepared according to the route outlined in Scheme 1) and acetaldehyde. ¹H NMR (250 MHz, CDCl³) δ:2.54 (3H, s), 2.57 (3H, s), 7.02 (H, d, J=8 Hz), 7.24-7.65 (1H, m), 7.47 (1H, t, J=8 Hz), 7.91 (1H, dd, J=8.5 and 1Hz), 8.41 (1H, d, J=1Hz), 8.59 (1H, d, J=8.5 Hz), NH not observed; m/z (API⁺): 308.1 (MH⁺).

### Examples 85-120

Prepared from 2-[5-(6-methylpyridin-2-yl)-4-quinoxalin-6-yl-1H-imidazol-2-yl]-methylamine according to the method of Examples 23-70.

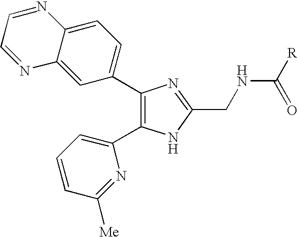

| | | m/z |
|---|---|---|
| Example | R | (API⁺) |
| 85 | 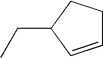 | 425 |
| 86 | benzyl | 435 |
| 87 | 3-chlorobenzyl | 470 |
| 88 | 4-fluorobenzyl | 453 |
| 89 | 4-methoxybenzyl | 465 |
| 90 | —(CH₂)₃—Ph | 463 |
| 91 | 4-nitrobenzyl | 480 |
| 92 | 4-dimethylaminobenzyl | 478 |
| 93 | cyclohexyl | 427 |
| 94 | n-propyl | 387 |
| 95 | —CH₂SPh | 467 |
| 96 | cinnamyl | 447 |
| 97 | n-heptyl | 443 |
| 98 | 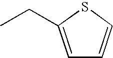 | 441 |
| 99 | 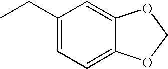 | 479 |
| 100 | 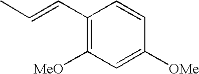 | 507 |
| 101 | 3-bromophenyl | 501 |
| 102 | 4-phenoxyphenyl | 513 |
| 103 | 4-methoxyphenyl | 451 |
| 104 | 4-acetylphenyl | 463 |
| 105 | 4-trifluorophenyl | 489 |
| 106 | 2-methoxy-4- | 497 |
| | methylsulfanylphenyl | |
| 107 | 4-n-pentyloxyphenyl | 507 |
| 108 | 3-thiophenyl | 427 |
| 109 | 1-methylindol-2-yl | 474 |
| 110 | benzofuran-2-yl | 461 |
| 111 | pyrazin-2-yl | 423 |
| 112 | 6-chloro-pyridin-3-yl | 456 |
| 113 | 6-methyl-pyridin-3-yl | 436 |
| 114 | 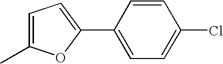 | 522 |
| 115 | 2-quinolyl | 521 |
| 116 | 3-methylbenzyl | 472 |
| 117 | 4-t-butylphenyl | 449 |
| 118 | 4-ethylphenyl | 477 |
| 119 | 2,3-dimethylphenyl | 449 |
| 120 | 2,6-dimethylphenyl | 449 |

### Examples 121-165

Prepared from 2-[4-(4-methoxyphenyl)-5-(6-methylpyridin-2-yl)-1H-imidazol-2-yl]-methylamine according to the method of Examples 23-70.

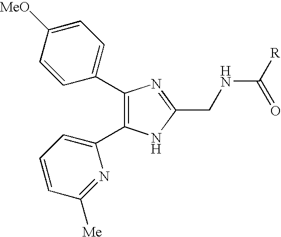

| | | m/z |
|---|---|---|
| Example | R | (API⁺) |
| 121 | 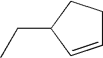 | 403 |
| 122 | benzyl | 413 |
| 123 | 3-chlorobenzyl | 447 |
| 124 | 4-fluorobenzyl | 431 |
| 125 | 4-methoxybenzyl | 443 |
| 126 | —(CH₂)₃—Ph | 441 |
| 127 | 4-nitrobenzyl | 458 |
| 128 | 4-dimethylaminobenzyl | 456 |
| 129 | cyclohexyl | 405 |
| 130 | n-propyl | 365 |
| 131 | —CH₂SPh | 445 |
| 132 | cinnamyl | 425 |
| 133 | n-heptyl | 421 |
| 134 | 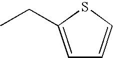 | 419 |
| 135 | indol-3-yl | 452 |
| 136 | 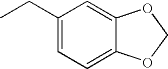 | 457 |
| 137 | 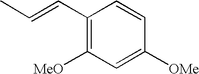 | 485 |
| 138 | 3-bromophenyl | 478 |
| 139 | 3,5-dichlorophenyl | 468 |
| 140 | 4-phenoxyphenyl | 491 |
| 141 | 4-methoxyphenyl | 429 |
| 142 | 4-phenylphenyl | 475 |
| 143 | 4-acetylphenyl | 441 |
| 144 | 4-trifluorophenyl | 467 |
| 145 | naphthyl | 449 |
| 146 | piperonyl | 443 |
| 147 | 3-nitrophenyl | 444 |
| 148 | 4-nitrophenyl | 444 |
| 149 | 2-methoxy-4- | 475 |
| | methylsulfanylphenyl | |
| 150 | 4-n-pentyloxyphenyl | 485 |
| 151 | 3-thiophenyl | 405 |
| 152 | 1-methylindol-2-yl | 452 |
| 153 | benzofuran-2-yl | 439 |
| 154 | pyrazin-2-yl | 401 |
| 155 | 6-chloro-pyridin-3-yl | 434 |
| | pyridin-4-yl | 400 |
| 156 | | |
| 157 | benzothiophen-2-yl | 455 |
| 158 | 2,6-dimethoxypyridin-3-yl | 460 |
| 159 | 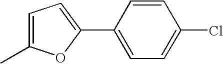 | 499 |
| 160 | 2-quinolyl | 450 |
| 161 | 3-methylbenzyl | 427 |
| 162 | 4-t-butylphenyl | 455 |
| 163 | 4-ethylphenyl | 427 |
| 164 | 2,3-dimethylphenyl | 427 |
| 165 | 2,6-dimethylphenyl | 427 |

### Example 166

### 6-[2-tert-Butyl-5-(6-methylpyridin-2-yl)-1H-imidazol-4-yl]-4H-benzo[1,4]oxazin-3-one

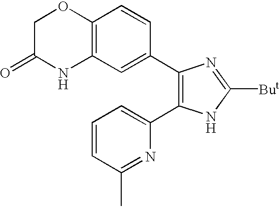

D13 (133mg, 0.3 mmol) was dissolved in acetic acid (2 ml). Iron powder (339 mg, 6 mmol) was added and the mixture stirred vigorously at 70° C. for 2 h. On cooling, the mixture was filtered through celite, washing with ethyl acetate. The solution was then evaporated to dryness and the residue partitioned between aq. sodium bicarbinate and ethyl acetate. The organic phase was dried over sodium sulfate, evaporated to dryness and the residue subjected to chromatography on silica gel eluting with 5% methanol in in ethyl acetate to afford the title compound (73 mg). ¹H NMR 250 MHz; DMSO-d⁶) Spectrum very broad due to restricted rotation on NMR timescale δ: 1.37 (9H, s), 2.49 (3H, s), 4.57 (2H, s), 6.80-7.31 and 7.63-7.57 (6H, m), 10.70 (1H, br.s), 11.80 (1H, br.s); m/z [ESMS]: 363.3 [M+H]⁺.

### Example 167

### 6-[2-tert-Butyl-5-(6-methylpyridin-2-yl)-1H-imidazol-4-yl]-4H-benzo[1,4]oxazine

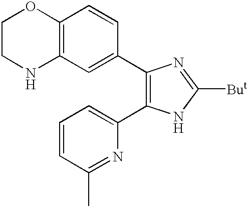

Prepared from Example 166 according to the procedure of Example 21. ¹H NMR (250 MHz; DMSO-d⁶) Spectrum broad due to restricted rotation on NMR timescale δ: 1.33 (9H, s), 2.43 (3H, s), 3.25 (2H, t), 4.10 (2H, t), 6.80-6.45 (3H, m), 7.00 (1H, d), 7.09 (1H, d), 7.50-7.41 (1H, m), NHs not observed; m/z [ESMS]: 349.3 [M+H]⁺.

### Example 168

### 6-[2-tert-Butyl-5-(6-methyl-pyridin-2-yl)-1H-imidazol-4yl]-quinoline

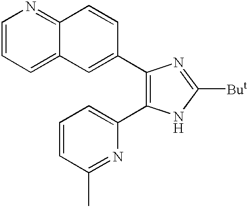

Prepared from 1-(6-methyl-pyridin-2-yl)-2-quinolin-6-yl-ethane-1,2-dione 1-oxime (prepared according to the route outlined in Scheme 1). ¹H NMR (250 MHz, CDCl₃) δ: 1.41 (9H, s), 2.37 (3H, s), 6.93 (1H, d, J=7.5 Hz), 7.21 (1H, d, J=8 Hz), 7.38-7.41 (2H, m), 7.92(1H, dd, J=9 and 2 Hz), 8.08 (1H, d, J=9 Hz), 8.16-8.18 (2H, m), 8.88-8.91 (1H, m), 11.41(1H, brs); m/z (API⁺): 343.3 (MH⁺).

Biological Data

The biological activity of the compounds of the invention may be assessed using the following assays:

Method for Evaluating ALK5 Kinase Phosphorylation of Smad3

Basic Flash-Plates (NEN Life Sciences) were coated by pipetting 100 micro liter of 0.1 molar sodium bicarbonate (pH 7.6), containing 150 nanograms of the fusion protein glutathion-S-transferase-smad3/100 micro liter of coating buffer. Plates were covered and incubated at room temperature for 10-24 hours. Then the plates were washed 2 times with 200 micro liter of coating buffer (0.1 molar sodium bicarbonate) and allowed to air dry for 2-4 hours.

For the phosphorylation reaction each well received 90 microliter containing 50 millimolar HEPES buffer (pH 7.4); 5 millimolar MgCl₂; 1 millimolar CaCl₂; 1 millimolar dithiothreitol; 100 micromolar guanosine triphosphate; 0.5 micro Ci/well gamma³³P-adenosine triphosphate (NEN Life Sciences) and 400 nanograms of a fusion protein of glutathion-S-transferase at the N-terminal end of the kinase domain of ALK5 (GST-ALK5). Background counts were measured by not adding any GST-ALK5. Inhibitors of ALK5 were evaluated by determining the activity of the enzyme in the presence of various compounds. Plates were incubated for 3 hours at 30° C. After incubation the assay buffer was removed by aspiration and the wells were washed 3 times with 200 microliter cold 10 millimolar sodium pyrophosphate in phosphate buffered saline. The last wash was aspirated and blotted plate dry. Plate was then counted on a Packard TopCount.

Fluorescence Anisotropy Kinase Binding Assay

The kinase enzyme, fluorescent ligand and a variable concentration of test compound are incubated together to reach thermodynamic equilibrium under conditions such that in the absence of test compound the fluorescent ligand is significantly (>50%) enzyme bound and in the presence of a sufficient concentration (>10×Kᵢ) of a potent inhibitor the anisotropy of the unbound fluorescent ligand is measurably different from the bound value.

The concentration of kinase enzyme should preferably be ≧1 ×K_{f}. The concentration of fluorescent ligand required will depend on the instrumentation used, and the fluorescent and physicochemical properties. The concentration used must be lower than the concentration of kinase enzyme, and preferably less than half the kinase enzyme concentration. A typical protocol is:

All components dissolved in Buffer of final composition 50 mM HEPES, pH 7.5, 1 mM CHAPS, 1 mM DTT, 10 mM MgCl₂ 2.5% DMSO.

ALK5 Enzyme concentration: 4 nM

Fluorescent ligand concentration: 1 nM

Test compound concentration: 0.1 nM-100 uM

Components incubated in 10 ul final volume in LJL HE 384 type B black microtitre plate until equilibrium reached (5-30 mins)

Fluorescence anisotropy read in LJL Acquest.

Definitions: Kᵢ=dissociation constant for inhibitor binding

K_{f}=dissociation constant for fluorescent ligand binding

The fluorescent ligand is the following compound:

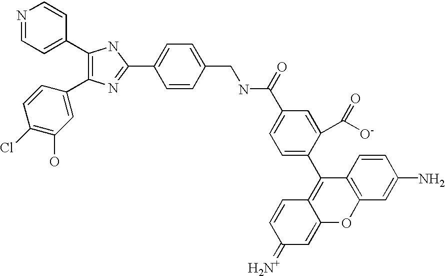

which is derived from 5-[2-(4-aminomethylphenyl)-5-pyridin-4-yl-1H-imidazol-4-yl]-2- chlorophenol and rhodamine green.

Inhibition of Matrix Markers: Northern Blot Protocol

Data confirming activity in the enzyme assay was obtained as follows.

A498 renal epithelial carcinoma cell lines were obtained from ATCC and grown in EMEM medium supplemented with 10% fetal calf serum, penicillin (5 units/ml) and streptomycin (5 ml). A498 cells were grown to near confluence in 100 mm dishes, serum-starved for 24 hours, pre-treated with compounds for 4 hours followed by a 10 ng/ml addition of TGF-betal (R&D Systems, Inc., Minneapolis Minn.). Cells were exposed to TGF-beta1 for 24 hours. Cellular RNA was extracted by acid phenol/chloroform extraction (Chomczynski and Sacchi, 1987). Ten micrograms of total RNA were resolved by agarose gel electrophoresis and transferred to nylon membrane (GeneScreen, NEN Life Sciences, Boston Mass.). Membranes were probed with 32P-labeled cDNA probes (Stratagene, La Jolla, Calif.) for fibronectin mRNA. Membranes were exposed to phosphorimaging plates and bands were visualized and quantified with ImageQuant software (Molecular Dynamics, Sunnyvale, Calif.).

Inhibition of Matrix Markers: Western Blot Protocol

Data confirming activity in the enzyme assay was obtained as follows.

Cells were grown to near confluence in flasks, starved overnight and treated with TGF-beta and compounds. Cells were washed at 24 or 48 hours after treatment with ice cold phosphate buffered saline, then 500 microliter of 2× loading buffer was added to plate and cells were scraped and collected in microcentrifuge tube. (2× loading buffer: 100 mM Tris-Cl, pH6.8, 4% sodium dodecyl sulfate, 0.2% bromophenol blue, 20% glycerol, 5% beta-mercapto-ethanol). Cells were lysed in tube and vortexed. Sample was boiled for 10 minutes. 20 microliters of sample was loaded on 7.5% polyacrylamide gel (BioRad) and electrophoresed.

Size fractionated proteins in gel were transferred to nitrocellulose membrane by semidry blotting. Membrane was blocked overnight with 5% powdered milk in phosphate buffer saline (PBS) and 0.05% Tween-20 at 4 degrees C. After 3 washes with PBS/Tween membranes were incubated with primary antibody for 4 hours at room temperature. After three washes with PBS/Tween membrane was incubated with secondary antibody for 1 hour at room temperature. Finally, a signal was visualized with ECL detection kit from Amersham.

The compounds of this invention generally show ALK5 receptor modulator activity having IC₅₀ values in the range of 0.0001 to 10 μM.

1. A compound of formula (I) or a pharmaceutically acceptable salt thereof: 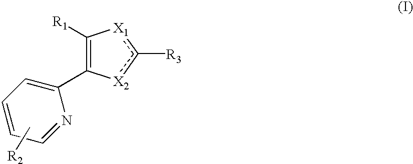
(I)
wherein R₁ is naphthyl, anthracenyl, or phenyl optionally substituted with one or more substituents selected from the group consisting of halo, C₁₋₆alkoxy, C₁₋₆alkylthio, C₁₋₆alkyl, C₁₋₆haloalkyl, O—(CH₂)ₘ—Ph, S—(CH₂)ₘ—Ph, cyano, phenyl, and CO₂R, wherein R is hydrogen or C₁₋₆alkyl and m is 0-3; or R₁ is phenyl or pyridyl fused with an aromatic or non-aromatic cyclic ring of 5-7 members wherein said cyclic ring optionally contains up to three heteroatoms, independently selected from N, O and S, and is optionally substituted by ═O;
R₂ represents hydrogen, C₁₋₆alkyl, C₁₋₆alkoxy, phenyl, C₁₋₆haloalkyl, halo, NH₂, NH—C₁₋₆alkyl or NH(CH₂)ₙ—Ph wherein n is 0-3;
R₃ represents C₁₋₆alkyl, —(CH₂)ₚ—CN, —(CH₂)ₚ—COOH, —(CH₂)ₚ—CONHR₄R₅, —(CH₂)ₚCOR₄, —(CH₂)_{q}(OR₆)₂, —(CH₂)ₚOR₄, —(CH₂)_{q}—CH═CH—CN, —(CH₂)_{q}—CH═CH—CO₂H, —(CH₂)ₚ—CH═CH—CONHR₄R₅, —(CH₂)ₚNHCOR₇ or —(CH₂)ₚNR₈R₉,
R₄ and R₅ are independently hydrogen or C₁₋₆alkyl;
R₆ is C₁₋₆alkyl;
R₇ is C₁₋₇alkyl, or optionally substituted aryl, heteroaryl, arylC₁₋₆alkyl or heteroarylC₁₋₆alkyl;
R₈ and R₉ are independently selected from hydrogen, C₁₋₆alkyl, aryl and arylC₁₋₆alkyl;
p is 0-4;
q is 1-4;
one of X₁ and X₂ is N and the other is NR₁₀; and
R₁₀ is hydrogen, C₁₋₆alkyl, or C₃₋₇cycloalkyl;
provided that the compound is not:
i) 2-[5-(2-methylphenyl)-2-propyl-1H-imidazol-4-yl]pyridine,
ii) 2-[2-(1,1-dimethylethyl)-5-(4-methoxyphenyl)-1H-imidazol-4-yl]pyridine,
iii) 2-[2-(1,1-dimethylethyl)-5-phenyl-1H-imidazol-4-yl]pyridine,
iv) 2-[5-(3,5-dichlorophenyl)-2-methyl-1H-imidazol-4-yl]pyridine,
v) 2-[5-(3,5-dimethylphenyl)-2-methyl-1H-imidazol-4-yl]pyridine,
vi) 2-[5-(3,5-dimethylphenyl)-2-ethyl-1H-imidazol-4-yl]pyridine,
vii) 2-[5-(3,5-dimethylphenyl)-2-amino-1H-imidazol-4-yl]pyridine,
viii) 2-[5-(3,5-dimethylphenyl)-2-isopropyl-1H-imidazol-4-yl]pyridine,
ix) 2-[5-(3,5-dimethylphenyl)-2-propyl-1H-imidazol-4-yl]pyridine,
x) 2-[5-(3,5-dimethylphenyl)-2-carboxamide-1H-imidazol-4-yl]pyridine,
xi) 2-[5-(3,5-dimethylphenyl)-2-cyano-1H-imidazol-4-yl]pyridine, or
xii) 2-[5-(3,5-dimethylphenyl)-2-methoxymethyl-1H-imidazol-4-yl]pyridine.

2. A compound according to claim 1 wherein R₁ is phenyl optionally substituted with one or more substituents selected from the group consisting of halo, C₁₋₆alkoxy, C₁₋₆alkylthio, and cyano; or R₁ is phenyl or pyridyl fused with an aromatic or non-aromatic cyclic ring of 5-7 members wherein said cyclic ring optionally contains up to three heteroatoms, independently selected from N, O and S, and is optionally substituted by ═O.

3. A compound according to claim 1 or 2 wherein R₂ is positioned ortho to the nitrogen of the pyridyl ring.

4. A compound according to any one of the preceding claims wherein R₃ is C₁₋₆alkyl or (CH₂)ₚNHCOR₇ wherein R₇ is C₁₋₇alkyl, or optionally substituted aryl, heteroaryl, arylC₁₋₆alkyl or heteroarylC₁₋₆alkyl.

5. A compound according to any one of the preceding claims wherein R₁₀ is hydrogen.

6. A compound according to claim 1 as defined in any one of Examples 1 to 71, or a pharmaceutically acceptable salt thereof.

7. A pharmaceutical composition comprising a compound according to any one of the preceding claims, but without provisos iv) to xii), or a pharmaceutically acceptable salt thereof, and a pharmaceutically acceptable carrier or diluent.

8. A method of inhibiting the TGF-β signaling pathway in mammals, comprising administering to a mammal, comprising administering to a mammal in need of such treatment, a therapeutically effective amount of a compound according to any one of claims 1 to 6, but without provisos i) to xii), or a pharmaceutically acceptable salt thereof.

9. A method for treating a disease selected from chronic renal disease, acute renal disease, wound healing, arthritis, osteoporosis, kidney disease, congestive heart failure, ulcers, ocular disorders, corneal wounds, diabetic nephropathy, impaired neurological function, Alzheimer's disease, trophic conditions, atherosclerosis, peritoneal and sub-dermal adhesion, any disease wherein fibrosis is a major component, and restenosis, comprising administering to a mammal in need of such treatment, a therapeutically effective amount of a compound according to any one of claims 1 to 6, but without provisos i) to xii), or a pharmaceutically acceptable salt thereof.

10. A method for inhibiting matrix formation in mammals, comprising administering to a mammal, a therapeutically effective amount of a compound according to any one of claims 1 to 6, but without provisos i) to xii), or a pharmaceutically acceptable salt thereof.
